(19) United States
(12) Patent Application Publication
(10) Pub. No.: US 2022/0025432 A1
(43) Pub. Date: Jan. 27, 2022

(54) METHOD FOR FLUORESCENCE LABELING OF BIOLOGICAL MATERIALS, THERMALLY REMOVABLE FLUORESCENT LABELS FOR THIS METHOD, AND METHODS OF THEIR PREPARATION AND USE

(71) Applicant: FUTURESYNTHESIS SPÓLKA Z O.O., Poznan (PL)

(72) Inventors: Marcin CHMIELEWSKI, Poznan (PL); Jolanta BRZEZINSKA, Swarzedz (PL); Agnieszka WITKOWSKA, Poznan (PL); Piotr PUTAJ, Kraków (PL)

(73) Assignee: FUTURESYNTHESIS SPÓLKA Z O.O., Poznan (PL)

(21) Appl. No.: 17/311,678

(22) PCT Filed: Dec. 3, 2019
(86) PCT No.: PCT/PL2019/000116

§ 371 Date: Jun. 7, 2021

(30) Foreign Application Priority Data
Dec. 6, 2018 (PL) P.428082
Dec. 6, 2018 (PL) P.428083
Dec. 6, 2018 (PL) P.428084

(51) Int. Cl. C12Q 1/6806; C12Q 1/6841; C07D 213/74; C09B 23/14; C09K 11/06

(52) U.S. Cl. CPC C12Q 1/6806; C12Q 1/6841; C07D 213/74; C09K 2211/1011; C09K 11/06; C09K 2211/1018; C09K 2211/1007; C09B 23/145

(57) ABSTRACT

A thermosensitive fluorescent label for labeling of biological material with fluorescent dyes used for biological, chemical or physical analyzes. A method for fluorescence labeling of nucleosides, nucleotides and oligonucleotides with those fluorescent dyes wherein a moiety of general formula 1 or 2 or 3 or 4 with the double bond configuration is attached to a nucleoside, nucleotide or oligonucleotide.

The present invention relates to a labeling method of biological material with fluorescent dyes to be used in biological, chemical and physical analyzes, as well as new fluorescent dyes for this method and methods of their preparation.

Many current analytical, diagnostic and synthetic techniques use the phenomenon of fluorescence. Among the most common techniques are FISH (Fluorescence in situ hybridization), RT-PCR (Real-time polymerase chain reaction), FRET (Forster resonance energy transfer), methods using single or double labeled molecular probes, fluorescence of amino acids, proteins, and quantum dots. The source of fluorescence is most often a dye, which in the labeling process is combined with another molecule, e.g. a biopolymer, most often a nucleic acid, peptide or protein.

The purpose of fluorescence labeling of biomolecules is to obtain information on the location of biomolecules in cells or organs, on physicochemical interactions or conformational changes, e.g. fluorescent in situ hybridization (FISH) is used to detect a specific DNA sequence in the given genetic material tested using specific probes labeled with fluorescent dyes. The fluorescence based methods are characterized by low invasiveness as well as a small negative impact on the operator's health and the environment, thus, in many cases, they are better suited than other labeling techniques, e.g. radioisotope labeling.

Known fluorescent dyes used to label biological material differ in their absorption and emission spectra. Moreover, the range of each spectrum represents only a narrow part of the visible light range. Consequently, there is no single dye for universal use with broad functionality. Therefore, sometimes to achieve the desired effect, a combination of several dyes must be used.

A fluorescent label is a fluorescent dye conjugated to any organic molecule, for example a biopolymer such as a nucleic acid, peptide or protein.

One of the methods used for fluorescence labeling of oligonucleotides, which is the most convenient and effective method of this kind, consists in conversion of the fluorescent dye into a phosphoramidite, which is then used to label an oligonucleotide by attaching to a hydroxyl, amino, or carboxyl group in an oligomer or in a part thereof.

The most popular fluorescent dyes available in the form of phosphoramidites include: fluorescein and its tetra- and hexachlorinated derivatives (TET and HEX). The advantage of these dyes is sufficient stability in an alkaline environment necessary for final unblocking of the oligonucleotide by removing protecting groups. Phosphoramidite derivatives are also used to label oligomers with fluorescent dyes such as cyanines. However, these compounds, when attached to the oligomer, may be unstable under basic conditions, and thus require the oligonucleotide unblocking conditions to be changed.

Another method of oligonucleotide fluorescence labeling is labeling within the oligonucleotide sequence.

In this type of oligonucleotide fluorescence labeling, it is possible to attach the fluorescent dye to the nitrogenous bases or the ribose ring of the respective nucleoside either during the synthesis of the oligonucleotide or after the oligomer synthesis.

In the first case, the dye is chemically converted into a phosphoramidite, which is subsequently attached to the free hydroxyl group of the oligonucleotide via activation with a 1-H-tetrazole derivative in course of chemical synthesis of the oligonucleotide by the phosphoramidite method on a solid support.

In the second case, the fluorescent dye is incorporated into the oligonucleotide in a post-synthetic process, i.e. after the synthesis of the oligonucleotide has been completed, the dye derivative is covalently bonded by reaction with a suitably modified nucleotide of the oligomer.

Examples of the post-synthetic labeling of oligomers are:
a reaction of the free amino group of one of the oligomer nucleotides with a N-hydroxysuccinimide ester or an isothiocyanate derivative of the dye. As a result of the reaction between such dye derivatives and the heterocyclic part of the oligomer a stable chemical bond is formed: an amide bond or a thiourea bond, respectively.
a reaction based on the so-called “click chemistry” between an oligonucleotide containing an alkyne modified nucleotide and a fluorescent dye containing an azido group. As a result of the reaction, a triazole ring is formed through the formation of carbon-nitrogen covalent bonds.

All state-of-the-art labeling methods presented above are based on the formation of a stable chemical bond between the dye and the nucleic acid.

In some situations, however, it is desirable for the fluorescent dye to be detachable from the nucleic acid without complete or partial degradation of the nucleic acid. This is important because even partial degradation of the nucleic acid results in a loss of its natural functions. For example, it is often observed that a dye attachment modifies the properties of a nucleic acid (i.e. some of its functions become blocked), which precludes the use of such a modified nucleic acid in accordance with its natural properties.

To date, no cases of safe removal of fluorescent labels from nucleic acids have been disclosed in the literature. The label-removing methods known to date suffer from destruction of the entire nucleic acid molecule occurring during the label detachment. For example, there are methods to remove fluorescent dyes from labeled nucleic acids by enzymatic digestion: the enzyme removes the phosphodiester linked components from the biopolymer step by step in the 5′ to 3′ direction. When at the 5′ end of such a biopolymer a fluorescent marker is attached, it will be removed first. However, the enzyme action cannot be stopped, which leads as a consequence to a complete degradation of the nucleic acid.

The objective of the invention is to develop a method for fluorescence labeling of biomolecules with a fluorescent tag and a method for thermal cleavage of the tag from the biomolecule leaving the biomolecule intact.

In the second aspect, the invention consists in a development of a fluorescent dye for the use as fluorescent tag, in particular for labeling nucleosides, nucleotides and oligonucleotides that can be detached from the labeled biomolecule without causing damage or degradation of this biomolecule.

The subject of the invention is a method for labeling of biological material, in particular nucleosides, nucleotides or oligonucleotides, with fluorescent dyes, which consists in an attachment of a moiety of general formula 1 or 2 or 3 or 4 with the double bond configuration E to nucleosides, nucleotides or oligonucleotides.

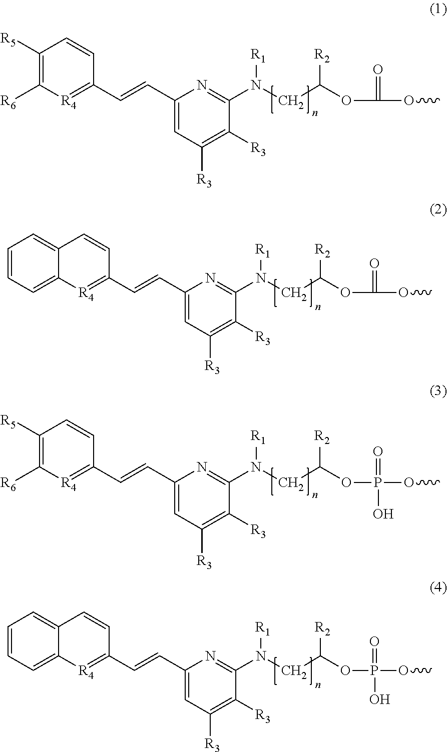

### Herein:

n is 1 or 2
[symbol] marks the bonding site with the nucleoside or nucleotide or oligonucleotide
R₁ means:
   hydrogen, methyl, amino group, carbonyl, benzyl, naphthyl, naphthylmethyl, phenyl, benzyl, quinolinemethyl,
   benzyl substituted with one or more the same or different substituents: chlorine, fluorine, methyl.
   saturated or unsaturated alkyl containing from 2 to 8 carbon atoms and one double bond, saturated alkyl containing from 2 to 8 carbon atoms substituted with a phenyl, amino group, hydroxyl group or simultaneously phenyl and hydroxyl group or phenyl and amino group,
R₂ means: hydrogen, methyl, benzyl, phenyl, naphthyl, saturated or unsaturated alkyl containing from 2 to 8 carbon atoms and one double bond,
R₃ are the same or different and represent hydrogen, methyl, saturated or unsaturated alkyl containing from 2 to 8 carbon atoms and one double bond, amino group, nitro group, azido group, a group of general formula 5
R₄ means carbon or nitrogen

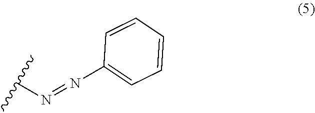 [SMILES: *N=Nc1ccccc1]

R₅ and R₆ are the same or different and represent hydrogen, methyl, benzyl, phenyl, naphthyl, halogen (F, Cl, Br, I), hydroxyl, amino group, nitro group, carboxyl, saturated or unsaturated alkyl containing 2 to 8 carbon atoms and one double bond, saturated or phenyl substituted unsaturated alkyl containing 2 to 8 carbon atoms and one double bond.
The label attachment occurs in a reaction of a free primary hydroxyl group of the nucleoside, nucleotide or oligonucleotide with a chemical compound of general formula 6 or 7 or 8 or 9

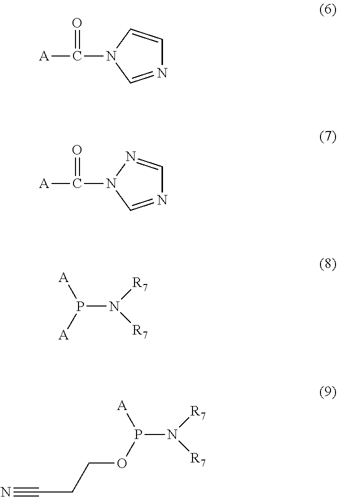

wherein A represents a group of general formula 10 or 11

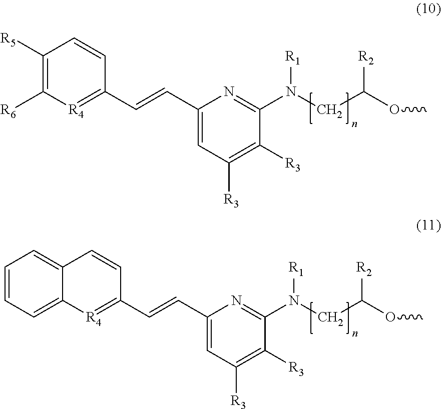

wherein n and substituents R₁.R₂. R₃.R₄. R₅, R₆ have the same meaning as given above, with the compound of the formula 6 or 7 being obtained directly in the reaction medium as reaction product between an alcohol of general formula 12 or 13

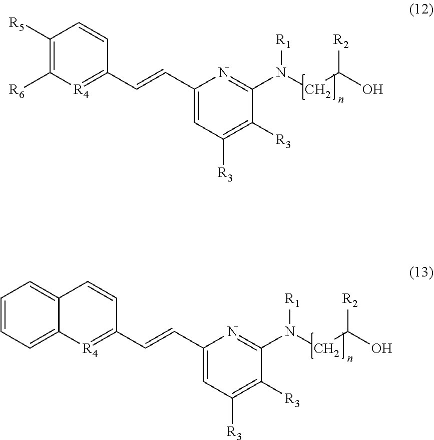

wherein n and substituents R₁, R₂, R₃, R₄, R₅, and R₆ have the same meaning as above, with a carbonyldimidazole of formula 14 or a carbonyldi(1,2,4-triazole) of formula 15

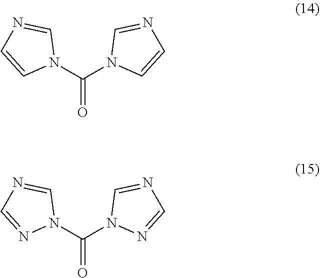 [SMILES: O=C(n1ccnc1)n1ccnc1 ; O=C(n1cncn1)n1cncn1]

forming intermediates of general formulas 6 or 7, with a compound of formula 8 being added into the reaction medium or being obtained directly in the reaction medium by reacting an alcohol of general formula 12 or 13, wherein n and substituents R₁, R₂, R₃, R₄, R₅, and R₆, have the same meaning as above, with N,N,N′,N′-bis(diisopropylamino)-chlorophosphine of formula 16,

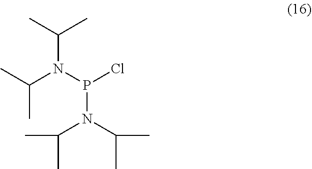

whereat the reaction between the alcohol of general formula 12 or 13 and N,N,N′,N′-bis(diisopropylamino)chlorophosphine proceeds in two steps: in the first step, N,N,N′,N′-bis(diisopropylamino)chlorophosphine reacts with one alcohol molecule of general formula 12 or 13, and in the second step, a second molecule of the alcohol is attached upon addition of 1-H-tetrazole or 2-ethylthiotetrazole or 4,5-dicyanoimidazole or 5-benzyl-mercaptotetrazole, and whereat the compound of formula 9 is injected into the reaction medium or is obtained directly in the reaction medium by reacting an alcohol of formula 12 or 13, wherein n and substituents R₁, R₂, R₃, R₄, R₅, and R₆ have the same meaning as above, with 2-cyanoethyl-N,N,N′,N′-bis(diisopropylamino)phosphine of formula 17

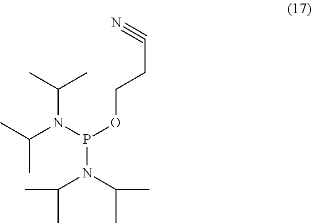 [SMILES: CC(C)N(C(C)C)P(OCCC#N)N(C(C)C)C(C)C]

in the presence of 1H-tetrazole or 2-ethylthiotetrazole or 4,5-dicyanoimidazole or 5-benzyl-mercaptotetrazole, wherein after the reaction between a nucleoside, nucleotide or oligonucleotide and the intermediate compound of the formula 8 or 9 is completed, the reaction product is subjected to oxidation with standard agents used in the oxidation of phosphorus (III) compounds, such as a solution of iodine in pyridine or ((1 S)-(+)-(10-camphorsulfonyl)-oxaziridine in acetonitrile and then, after oxidation, methylamine or 1,8-diazabicyclo[5.4.0]undec-7-ene (DBU) or concentrated aqueous ammonia solution is added in an amount necessary to complete the synthesis of the compound of the formula 3 or 4.

Generalized embodiments of the method being subject of the invention using various types of intermediates are presented in the following examples.

### Labeling Method Using Active Imidazole Derivatives of Formula 6.

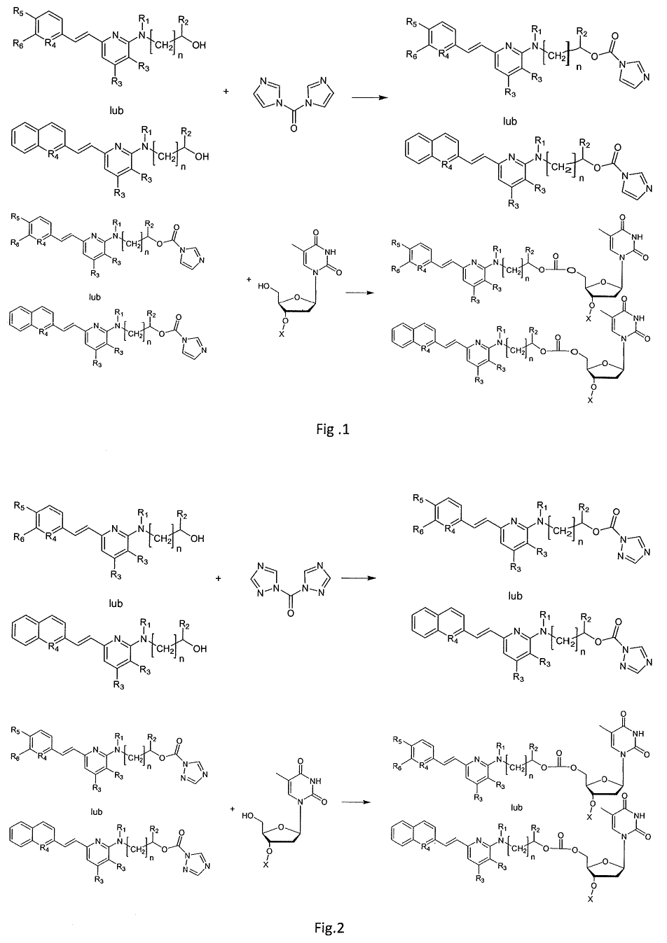

The procedure is schematically illustrated in FIG. 1.

An alcohol of general formula 12 or 13 is dissolved in anhydrous polar aprotic organic solvent selected from the group consisting of acetonitrile, dimethyl sulfoxide, dimethylformamide, aliphatic ethers, in particular diethyl ether and tetrahydrofuran, ketones, in particular acetone, preferably in acetonitrile or tetrahydrofuran. To this solution a carbonyldiimidazole is added in the proportion of 1 to 1.5 eq., preferably 1.3 eq., relative to the alcohol used. The reaction is carried out at ambient temperature, preferably at 25° C., but not higher than 30° C. The reaction time is between 30 minutes and 2 hours, preferably 1 hour. After this time, an active imidazole derivative is formed, which is added to the nucleoside, nucleotide or oligonucleotide (with a free hydroxyl or amino group) in an excess of 1 to 2 eq., preferably 1.5 eq. The reaction is carried out in the presence of a non-nucleophilic organic base selected from the group consisting of guanidine derivatives containing the same or different substituents selected from methyl, ethyl, isopropyl, preferably tetramethylguanidine (TMG) in relative amounts of 0.3-7.8 eq., preferably 3.5 eq. at ambient temperature, preferably at 25° C., but not higher than 30° C. After the reaction is completed, the solvent is removed and the product is isolated by standard methods.

An example of such a nucleoside labeled with an active imidazole derivative of formula 6 is a compound of formula 18

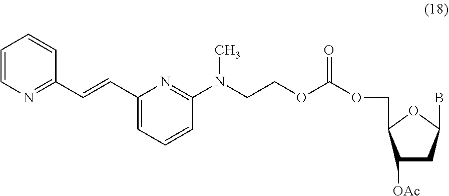

wherein B is a heterocyclic base e.g. thymine.

Labeling Method Using Active Imidazole Derivatives of the Formula 7.

The procedure is schematically illustrated in FIG. 1.

An alcohol of general formula 12 or 13 is dissolved in anhydrous polar aprotic organic solvent selected from the group consisting of acetonitrile, dimethyl sulfoxide, dimethylformamide, aliphatic ethers, in particular diethyl ether and tetrahydrofuran, ketones, in particular acetone, preferably in acetonitrile or tetrahydrofuran. To this solution carbonyldi(1,2,4-triazole) is added in a proportion of 1 to 1.5 eq. preferably, 1.3 eq., relative to the alcohol used. The reaction is carried out at ambient temperature, preferably at 25° C., but not higher than 30° C. The reaction time is between 10 minutes and 45 minutes, preferably 30 minutes. After this time, an active triazole derivative is formed which is added to the nucleoside, nucleotide or oligonucleotide (with a free hydroxyl group) in an excess of 1 to 2 eq. preferably 1.5 eq. The reaction is carried out at ambient temperature, preferably at 25° C., but not higher than 30° C. After the reaction is completed, the solvent is removed and the product is isolated by standard methods.

An example of a nucleoside labeled with an active triazole derivative of formula 7, is a compound of formula 18

Labeling Method Using Phosphoramidites:

Labeling method of a nucleoside, nucleotide or oligomers using a phosphoramidite of formula 8.

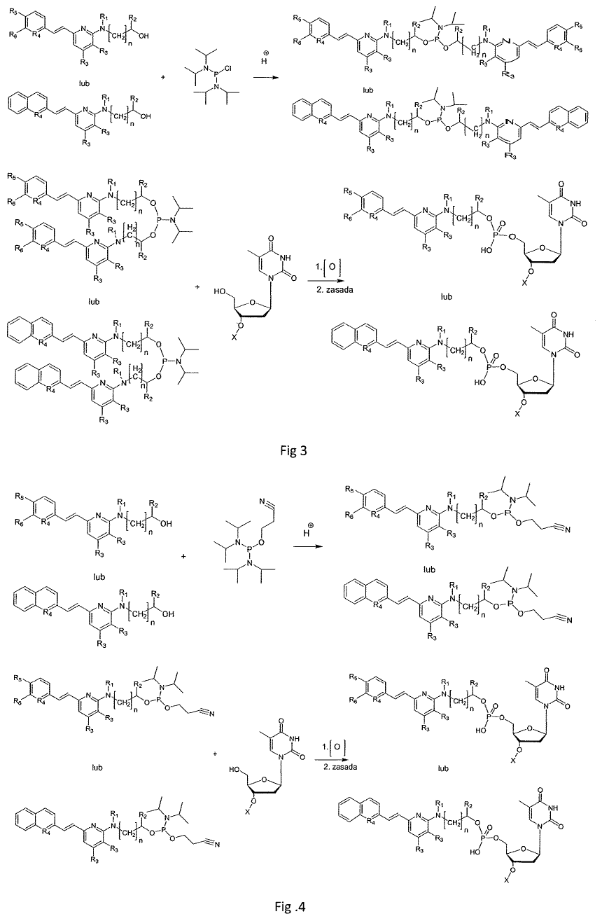

The procedure is schematically illustrated in FIG. 3.

Step I: Preparation of Phosphoramidite of Formula 8

An alcohol of formula 12 or 13 (2 eq.) is dissolved in anhydrous aprotic organic solvent, preferably in acetonitrile or methylene chloride, under an inert gas atmosphere, preferably argon. The whole procedure is carried out at a temperature in the range of 15-30° C., preferably at 20° C. Then, an anhydrous aliphatic tertiary amine, preferably diisopropylethylamine, is added to the solution to bind chlorine that is released in course of the reaction, in an amount of not less than 1 eq., preferably 1.3 eq. Then, N,N,N′,N′-bis(diisopropylamino)chlorophosphine is added to the solution in an amount of 0.8 to 1.1 eq., preferably 1 eq. The course of the reaction is monitored, preferably by a thin-layer plate chromatography (TLC) using benzene:triethylamine (9:1) as moving phase. After it has been established that half of the starting compound reacted, 1-H-tetrazole or 2-ethylthiotetrazole or 4,5-dicyanoimidazole or 5-benzyl-mercaptotetrazole is added in an amount of 0 to 0.8 eq., preferably 0.5 eq. After completion of the reaction, the solvent is removed and the product of the formula 8 is isolated by standard methods and purified by means of chromatographic methods in the presence of a tertiary aliphatic amine, preferably triethylamine.

Step II: Labeling of Nucleosides or Nucleotides or Oligomers

The thus obtained and purified a phosphoramidite of formula 8 is dissolved in anhydrous aprotic organic solvent, preferably acetonitrile, methylene chloride or toluene and mixed with 177-tetrazole or 2-ethylthiotetrazole or 4,5-dicyanoimidazole or 5-benzyl-mercaptotetrazolein in an amount of not less than 1 eq. Then, a nucleoside or nucleotide or oligonucleotide with a free hydroxyl group is added to the mixture. For a reaction carried out in liquid phase, an equimolar mixture of the phosphoramidite of formula 8 and 177-tetrazole or 2-ethylthiotetrazole or 4,5-dicyanoimidazole or 5-benzyl-mercaptotetrazole is used in a molar ratio of 2 eq. to 1 eq. relative to the nucleoside or nucleotide or oligonucleotide, while for reactions carried out on a solid phase (porous glass), a molar ratio of 20 eq. to 1 eq. is applied. The reaction time also depends on the methodology used amounting to between 0.5 and 3 hours, preferably 1 hour, for a liquid phase reaction and 0.5 to 6 minutes, preferably 3 minutes, for a solid phase reaction. After the phosphoramidite has been attached to a nucleoside or nucleotide or oligonucleotide, an oxidation reaction is carried out using standard agents used in the oxidation of phosphorus (III) compounds, such as a solution of iodine in pyridine or ((1S)-(+)-(10-camphorsulfonyl)-oxaziridine (CSO) in acetonitrile. Then, after the oxidation is completed, methylamine or 1,8-diazabicyclo[5.4.0]undec-7-ene (DBU) or concentrated aqueous ammonia solution is added in an amount necessary for the synthesis of a compound of formula 3 or 4 to be completed. As a result of the above reaction, a labeled nucleoside or nucleotide or oligonucleotide is obtained. After the reaction is completed, the solvent is removed by standard methods used for the respective reaction methodology (solid or liquid phase) applied and the product is isolated by standard methods.

In another form of embodiment, after completion of the reaction of step I, the product (of the formula 8) is not isolated but instead a nucleoside or nucleotide or oligonucleotide having a free hydroxyl group and 1H-tetrazole or 2-ethylthiotetrazole or 4,5-dicyanoimidazole or 5-benzyl-mercaptotetrazolein in an amount of not less than 1 eq. are added to the reaction mixture. Further, the process is carried out as described above for step II.

Labeling Method of a Nucleoside, Nucleotide or Oligomers Using a Phosphoramidite of the Formula 9,

The procedure is schematically illustrated in FIG. 4.

Step I: Preparation of Phosphoramidites of the Formula 9.

An alcohol of formula 12 or 13 is dissolved in anhydrous aprotic organic solvent, preferably in acetonitrile or methylene chloride, under an inert gas atmosphere, preferably argon. Then, 2-cyanoethyl-N,N,N′,N′-bis(diisopropylamino)phosphine is added to the solution in an amount of 1 to 3 eq. relative to the alcohol 12 or 13, preferably 1.3 eq., and 1H-tetrazole or 2-ethylthiotetrazole or 4,5-dicyanoimidazole or 5-benzyl-mercaptotetrazole is added in an amount not exceeding 1 eq., preferably 0.8 eq., at the temperature of 15-30° C., preferably 20° C. After completion of the reaction, the solvent is removed and the product being phosphoramidite of formula 9 is isolated by standard methods. For purification of the final product, a tertiary aliphatic amine, preferably triethylamine, is used.

Step II: Labeling of Nucleoside or Nucleotide or Oligomers

The thus obtained and purified phosphoramidite of formula 9 is dissolved in anhydrous aprotic organic solvent, preferably in acetonitrile, methylene chloride or toluene and mixed with 1H-tetrazole or 2-ethylthiotetrazole or 4,5-dicyanoimidazole or 5-benzyl-mercaptotetrazolein in an amount of not less than 1 eq. Then, the mixture is added to a nucleoside or nucleotide or oligonucleotide having a free hydroxyl group. In a reaction carried out in liquid phase, an equimolar mixture of a phosphoramidite of formula 9 and 1H-tetrazole or 2-ethylthiotetrazole or 4,5-dicyanoimidazole or 5-benzyl-mercaptotetrazole is applied in an amount of 2 eq. relative to the nucleoside or nucleotide or oligonucleotide being labeled, while in a reaction carried out on a solid phase (porous glass) a molar ratio of 20 eq. relative to the nucleoside or nucleotide or oligonucleotide being labeled is applied. Also, the reaction time depends on the methodology used and amounts to 0.5 up to 3 hours, preferably 1 hour, for a liquid phase reaction and 0.5 to 6 minutes, preferably 3 minutes, for a solid phase reaction. After the phosphoramidite 9 has been attached to a nucleoside or nucleotide or oligonucleotide, an oxidation reaction is carried out using standard agents used in the oxidation of phosphorus (III) compounds, such as the iodine pyridine solution or CSO in acetonitrile, preferably an aqueous solution of iodine in pyridine. Then, after the oxidation is completed, methylamine or 1,8-diazabicyclo[5.4.0]undec-7-ene (DBU) or concentrated aqueous ammonia solution is added in an amount necessary for the synthesis of a compound of formula 3 or 4 to be completed. As a result of the above reaction, a labeled nucleoside or nucleotide or oligonucleotide is obtained. Then, the solvent is removed by standard methods used for the respective reaction methodology (solid or liquid phase) applied and the product is isolated by standard methods.

An example of a thus labeled nucleoside obtained using an active phosphoramidite derivative of formula 8 or 9 is the compound of formula 19

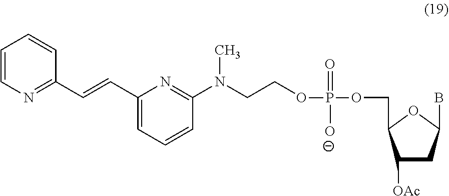

wherein B is a heterocyclic base e.g. thymine.

In another form of embodiment, after completion of the reaction of step I, the product (of formula 16) is not isolated but instead a nucleoside or nucleotide or oligonucleotide having a free hydroxyl group and 1H-tetrazole or 2-ethylthiotetrazole or 4,5-dicyanoimidazole or 5-benzyl-mercaptotetrazole in an amount of not less than 1 eq. are added to the reaction mixture. Further, the process is carried out as described above for step II.

The invention relates also to a method of thermal removal of fluorescent labels from a labeled nucleoside, nucleotide or oligonucleotide, resulting in a delabeled nucleoside, nucleotide or oligonucleotide along with a cyclic compound of general formula 20, 21 or 22.

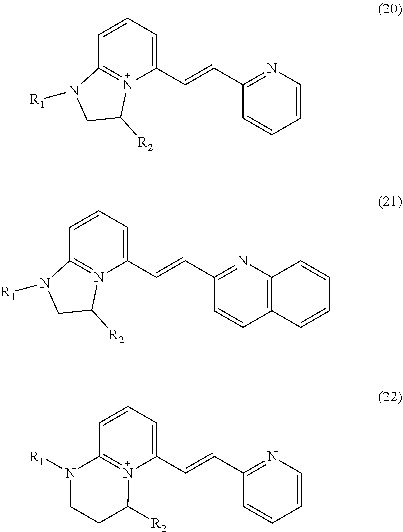

The label detachment is carried out as follows: the labeled nucleoside, nucleotide or oligonucleotide is dissolved in aqueous phosphate buffer (with a pH in the range of 6.86 to 7.2) and if the compound has not dissolved completely, acetonitrile is added to the phosphate buffer in an amount necessary to obtain a clear solution. The solution is then heated in a closed vessel to a temperature in the range of 30-90° C.

Scheme 1 shows an example of thermal removal of a fluorescent label from a nucleoside labeled with an imidazole derivative and Scheme 2 shows an example of thermal removal of a fluorescent label from a nucleotide labeled with a phosphoramidate derivative and the mechanisms of formation of five-membered bicyclic dye species, respectively.

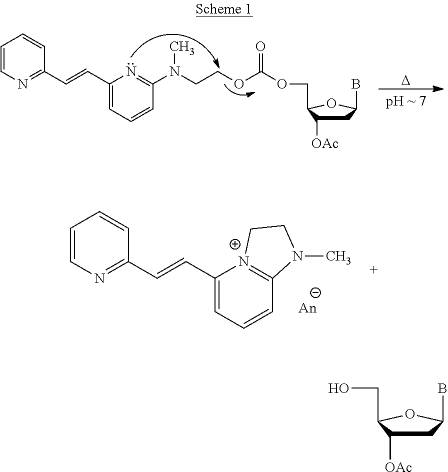

In Scheme 1, B is a heterocyclic base e.g. thymine and An is any anion present in the solution, preferably phosphate.

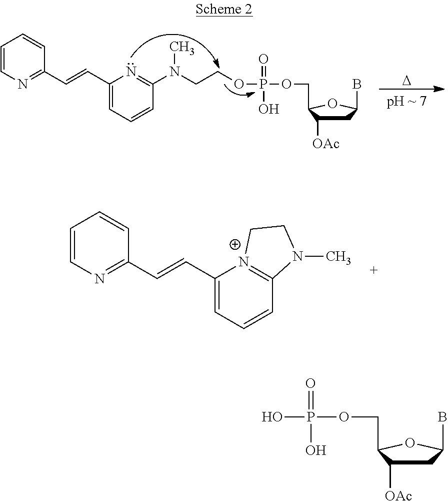

In Scheme 2, B is a heterocyclic base, e.g. thymine.

The conditions under which the particular label is detached are characteristic of a given label and differ from other labels, therefore, before using a given label, the parameters needed for its complete removal should be determined by plotting a calibration curve that describes the dependence of the label detachment on temperature and/or time, which will thus allow for proper adjustment of both the reaction temperature and reaction time needed for detachment of the particular label.

The calibration curve is characteristic for a given label. It does not depend, however, on the labeling method applied to attach the dye (the phosphoramidite-based method or the imidazole/triazole-based method)) and the type of the compound to which the dye is attached.

In the following, an example procedure for making a standard curve is described. First, the preferable temperature for the detachment of the label is determined. For this purpose, a labeled biomolecule, e.g. a nucleoside, nucleotide or oligonucleotide labeled with a given dye, is divided into 7 separate portions, 1 milligram each, and dissolved in phosphate buffer with a pH in the range of 6.86 to 7.2 or in a mixture of phosphate buffer with acetonitrile, preferably in a proportion of 2 parts by volume of the buffer to 1 part by volume of acetonitrile in a sealable vial. Then, each portion is heated for 2 hours at various temperatures: 30, 40, 50, 60, 70, 80, 90° C. under continuous shaking. The samples are subsequently subjected to RP-HPLC analysis with fluorescence detection, and a temperature dependence curve based on the chromatograms showing the degree of removal of the label from the nucleoside, nucleotide or oligonucleotide is drawn. Based on the curve, the temperature for which the time of complete label removal will be determined is chosen.

To determine the time needed for complete removal of the label at the temperature determined in previous step, it is proceeded as follows: 1 mg of a labeled biomolecule e.g. nucleoside, nucleotide or oligonucleotide labeled with a given dye is dissolved in phosphate buffer (pH 6.86 or 7.2) or in a mixture: phosphate buffer (pH 6.86 or 7.2)/acetonitrile (2/1; v/v) in a sealable vial. Then, it is heated at the previously determined temperature under continuous shaking. Aliquots taken from the sample after 5, 15, 30, 45, 60, 75, 90 minutes are analyzed by RP-HPLC with fluorescence detection. Based on the chromatograms showing the degree of the label removal, a curve describing the dependence of the label decay on time is plotted. Based on the curve, the time needed to completely remove the label from the nucleoside, nucleotide or oligonucleotide at a given temperature is then determined.

The progress of the label detachment and formation of the cyclic dye species can be monitored using a calibration curve obtained by spectrophotometric methods, preferably by use of RP-HPLC with a fluorescence detection that enables to detect the cyclic dye species formed. An example reaction of label removing from a nucleoside with formation of a cyclic dye species when the label is detached from a labeled nucleoside, nucleotide or oligonucleotide is shown in Scheme 1.

For example, when a dye of general formula 23 is used as the fluorescent label, after the cycle consisting of: (i) precursor formation, (ii) label attachment to a nucleoside, nucleotide, or oligonucleotide, and (iii) detachment of the label, a five-membered bicyclic compound of general formula 20 is formed. Similarly, when a dye of general formula 24 is used as the fluorescent label, after the cycle consisting of: (i) precursor formation, (ii) label attachment to a nucleoside, nucleotide, or oligonucleotide, and (iii) detachment of the label, a five-membered bicyclic species of formula 21 is formed.

When a dye of formula 25 is used as the fluorescent label, after the cycle consisting of: (i) precursor formation, (ii) label attachment to a nucleoside, nucleotide, or oligonucleotide, and (iii) detachment of the label, a five-membered bicyclic compound of formula 22 is formed.

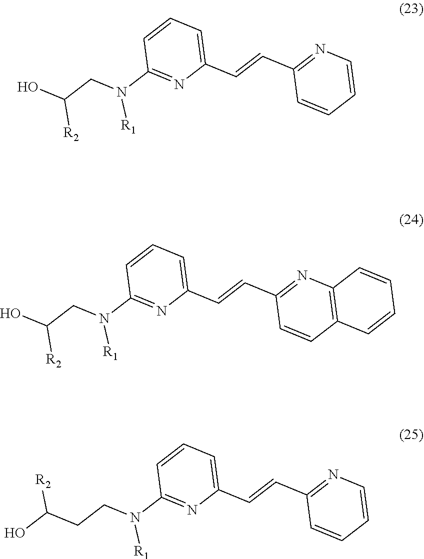

In the fourth aspect, the invention relates to a method of determination of the degree of fluorescent marker detachment from a labeled biomolecule consisting in measuring the difference in the fluorescence intensity of the labeled molecule, in the wavelength range corresponding to the emission of the label and the fluorescence intensity after detachment of the label from the labeled biomolecule at elevated temperature measured at the same wavelength range, corresponding to the label emission (FIG. 8).

The method consists in comparing the fluorescence intensity of the labeled molecules measured at the emission wavelength range corresponding to the label emission for the labeled biomolecules upon completion of the labeling procedure and the fluorescence intensity measured at the same wavelengths (measuring points) after selected intervals during the label detachment procedure. The thus collected data are analyzed numerically and based on the results of this analysis the decay dynamics and the degree of label detachment are determined, as well as the influence of external factors on the bond stability between the label and the biomolecule is assessed.

In order to determine the progress of the label detachment from the biomolecule, and thus the actual labeling level, the fluorescence intensity is measured for the labeled biomolecules in the sample that has not been heated and after removing the label at elevated temperature using a fluorescence detector for a 1 millimolar solution of the labeled biomolecule dissolved in such solvent or solvent mixture (e.g. ACN, DMF, phosphate buffer, citrate buffer) and at such elevated temperature for which the time of complete removal of the label is to be determined. The measurement is done at an emission wavelength within the range of the emission spectrum of the respective labeled biomolecule. The measurement is carried out at time intervals (preferably at 6 points) and a curve for the fluorescence intensity dependence on time for the labeled biomolecule is plotted. Thus, a curve describing changes of the fluorescence intensity/wavelength of the labeled biomolecule in time at a given temperature is determined. Using the thus determined curve for the emission intensity changes one can quantify the actual degree of the label detachment.

In the fifth aspect, the invention relates to new derivatives of pyridin-2-yl-vinylpyridine of general formula 12 or 13

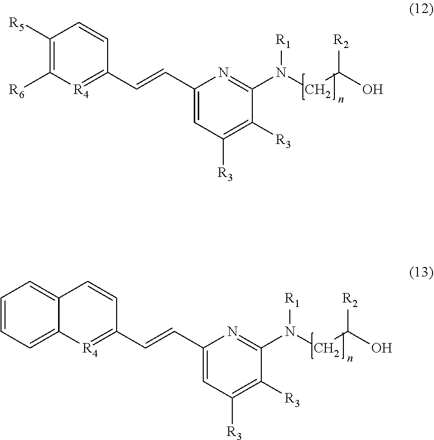

wherein:
n is 1 or 2
R₁ means:
   hydrogen, methyl, amino group, carbonyl, benzyl, naphthyl, naphthylmethyl, phenyl, benzyl, quinolinemethyl,
   benzyl substituted with one or more the same or different substituents: chlorine, fluorine, methyl.
   saturated or unsaturated alkyl containing from 2 to 8 carbon atoms and one double bond, saturated alkyl containing from 2 to 8 carbon atoms substituted with a phenyl, amino group, hydroxyl group or simultaneously phenyl and hydroxyl group or phenyl and amino group,
R₂ means: hydrogen, methyl, benzyl, phenyl, naphthyl, saturated or unsaturated alkyl containing from 2 to 8 carbon atoms and one double bond,
R₃ are the same or different and represent hydrogen, methyl, saturated or unsaturated alkyl containing from 2 to 8 carbon atoms and one double bond, amino group, nitro group, azido group, a group of general formula 5
R₄ means carbon or nitrogen

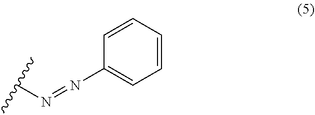 [SMILES: *N=Nc1ccccc1]

R₅ and R₆ are the same or different and represent hydrogen, methyl, benzyl, phenyl, naphthyl, halogen (F, Cl, Br, I), hydroxyl, amino group, nitro group, carboxyl, saturated or unsaturated alkyl containing 2 to 8 carbon atoms and one double bond, saturated or phenyl substituted unsaturated alkyl containing 2 to 8 carbon atoms and one double bond,

In the sixth aspect, the invention relates to a method of preparation of pyridin-2-yl derivatives of general formula 12 or 13, wherein n and substituents R₁, R₂, R₃, R₄, R₅, and R₆ have the same meanings as defined above.

The method of preparation of compounds of general formula 12 or 13, wherein substituents R₁, R₂, R₃, R₅, and R₆ have the same meanings as defined above and R₄ is carbon or nitrogen, consists in a reaction between a compound of general formula 26 or 27

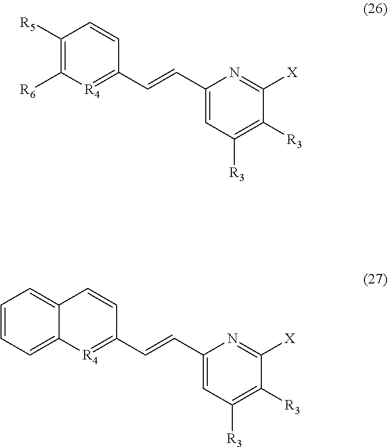

wherein R₃, R₄, R₅, and R₆ have the same meanings as defined above with an amino alcohol of general formula 28

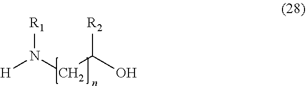

wherein R₁ and R₂ have the same meanings as defined above in the presence of a tertiary organic amine, preferably diisopropylethylamine in an anhydrous aprotic polar organic solvent, preferably DMF, preferably using an excess of the amino alcohol of the formula 28 of 1.5-5 eq., preferably 2 eq., relative to the compound 26 or 27. The reaction is carried out at an elevated temperature in the range of 70-120° C., preferably at 100° C., preferably using microwave heating of a frequency in the range of 300 GHz to 1 GHz and power in the range from 200 to 450 W. After the reaction is completed, the solvent is removed and the product is isolated by standard methods. The final product is a compound of general formula 12 or 13.
In a further aspect, the invention relates to a method of preparation of pyridin-2-yl-vinylpyridinyl (PvP) derivatives of general formula 12 or 13
wherein n and substituents R₁, R₂, R₃, R₄, R₅, and R₆ have the same meanings as defined above, consists in a reaction between a compound of general formula 29

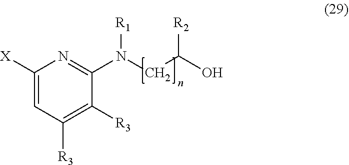

wherein substituents R₁, R₂, and R₃ have the same meanings as defined above and X is F, Cl, Br or I
with a compound of general formula 30 or 31

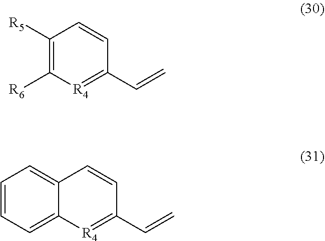

wherein R₅ and R₆ have the same meanings as defined above and R₄ is carbon or nitrogen in the presence of a catalytic complex formed from a palladium (II) salt.

The reaction is carried out according to the following procedure: a palladium (II) salt, preferably Pd(OAc)₂ is transferred into a polar aprotic organic solvent, preferably anhydrous DMF, in an amount of 0.01 eq. up to 0.1 eq., preferably 0.02 eq., together with a phosphine, preferably PPh₃, in an amount of 0.03 eq. up to 0.1 eq., preferably 0.06 eq., relative to the palladium salt and optionally with an addition of an ionic liquid such as tetrabutylammonium bromide (TBAB), tetrabutylammonium acetate (TBAA), 1-butyl-3-methylimidazolium bromide [BMIM][Br], 1-butyl-3-methylimidazolium hexafluorophosphate [BMIM][PF₆], tetraheptylammonium bromide (THPAB) in an amount from 0.03 eq. to 0.1 eq., preferably TBAB 0.06 eq. relative to the palladium salt. The reaction can also be carried out in the presence of palladium (II) salts, preferably Pd(OAc)₂ in an amount of 0.01 eq. up to 0.1 eq., preferably 0.02 eq., and an ionic liquid such as TBAB, TBAA, [BMIM][Br], [BMIM][PF₆], THPAB, in an amount of 0.03 eq. up to 0.1 eq., preferably TBAB 0.25 eq. relative to the palladium salt.

After a yellow suspension has formed, which generally occurs after at least 5 minutes, compounds 30 or 31 are added into the reaction mixture in a ratio of 0.6 eq. to 1.5 eq., preferably 1 eq., or from 0.6 eq. to 1.5 eq., preferably 1.2 eq., respectively, and a compound of formula 29

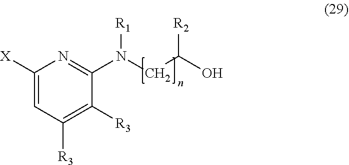

wherein R₁, R₂, and R₃ have the same meanings as defined above and X is halogen (F, Br, Cl, I), preferably Br, and a base in excess of 1.1 eq. do 1.8 eq., preferably 1.5 eq., relative to the compound 29, are added The base is selected from the group consisting of: tBuOLi, tBuOK, Cs₂CO₃, K₂CO₃, triethylamine (TEA), 1,8-diazabicyclo[5.4.0]undec-7-ene (DBU), KOH, NBu₄OH, NaOAc, K₃PO₄, preferably Cs₂CO₃. The reaction is carried out under an inert gas atmosphere, preferably argon, at a temperature not lower than 70° C., preferably 120° C., using heating, preferably microwave heating of the frequency in the range of 300 GHz to 1 GHz and power in the range from 200 to 450 W, for 2-14 hours, preferably 3 hours. After the reaction is completed, the solvent is removed and the product is isolated by standard methods. The reaction products are compounds of general formulas 12 or 13.

Determination of the absorption and emission properties of the fluorophores

### 1. Determination of the Absorption and Fluorescence Maxima

The subject of the investigation were compounds of formulas 32 and 33 with the double bond E configuration

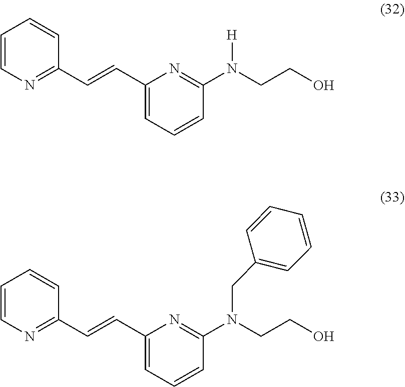 [SMILES: OCCNc1cccc(/C=C/c2ccccn2)n1 ; OCCN(Cc1ccccc1)c1cccc(/C=C/c2ccccn2)n1]

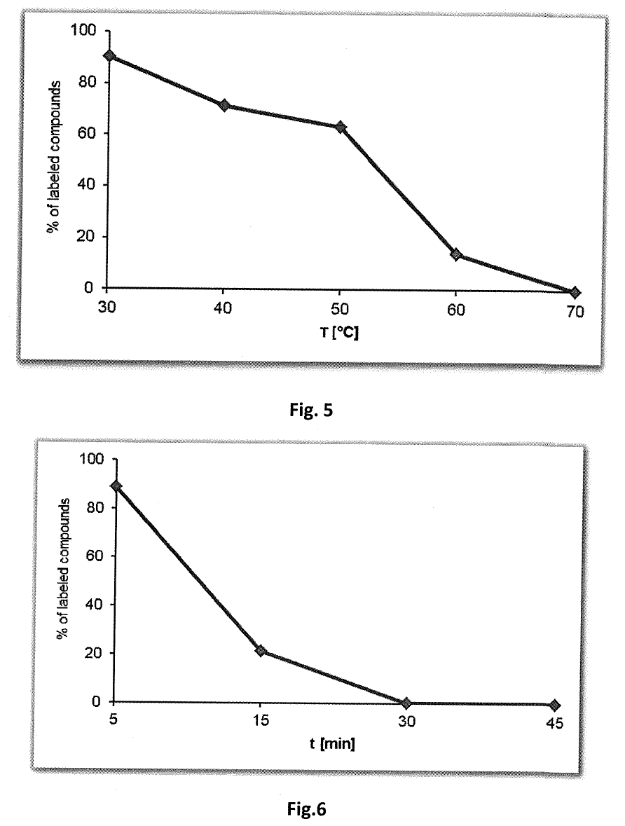

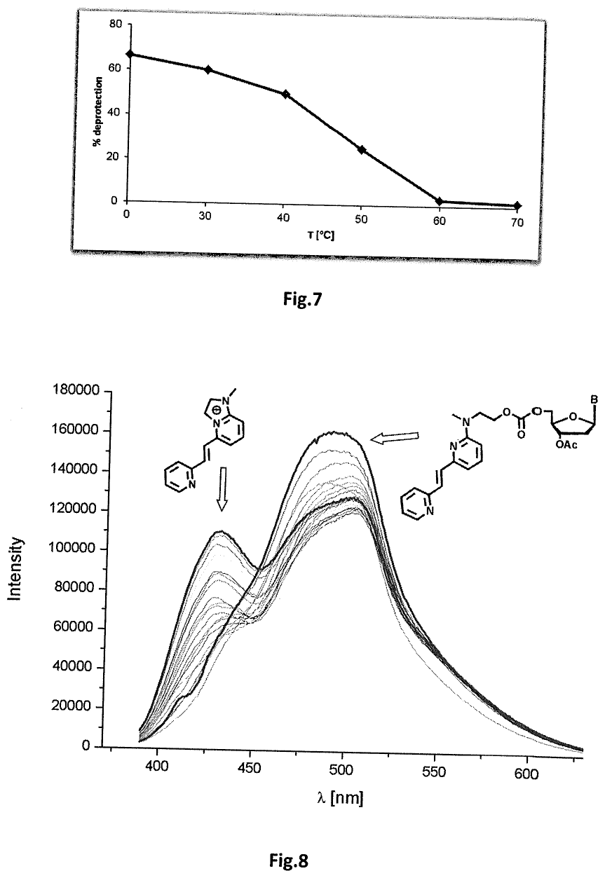

UV-vis absorption spectra were recorded for methanol solutions of compounds 32 and 33 with concentration of Cₘ=10⁻⁴ M in quartz cuvettes with an optical path of 1 cm in the spectral range of 220 nm to 700 nm, using a UV-Vis spectrophotometer (Cary 100, Agilent, USA). The absorption maxima determined for the two compounds from the thus recorded absorption spectra are 357 nm for compound 32 and 370 nm for compound 33, respectively. The spectra are shown in FIG. 5 (compound 32) and FIG. 6 (compound 33), respectively. Then, emission spectra of the two compounds were recorded using a FluoTime 300 EasyTau spectrofluorimeter (PicoQuant, Germany) at an excitation wavelength corresponding to the absorption maximum of the respective compound: 357 nm for compound 32 and 370 nm for compound 33, respectively. For compound 32, the emission maximum occurs at 465 nm (FIG. 7) and for compound 33, the emission maximum occurs at 470 nm (FIG. 8), respectively.

### 2. Determination of the Molar Absorption Coefficient [ε]

To determine the molar absorption coefficients ε of compounds 32 and 33, for each of the compounds 3 samples were weighted within an accuracy of 0.00001 g: 0.00059 g, 0.00045 g, and 0.00074 g for compound 32 and 0.00076 g, 0.00088 g, 0.00067 g for compound 33, respectively. Each sample was dissolved in 1 mL of pure methanol and the resulting solutions were placed in 5 mL volumetric flasks. Then 4 mL methanol was added to each of the volumetric flasks. For each 510 of the thus prepared solutions, the maximum absorbance value was measured in a quartz cuvette with an optical path of 1 cm and the ε values [M⁻¹ cm⁻¹] were determined according to Lambert-Beer law: A=εlc, where: A—absorbance, l—optical path length, c—molar concentration [mol/L], The measured values were averaged assuming an error margin of +/−20%. The results are listed in Table 1 in the second column.

### 3. Determination of the Fluorescence Quantum Yields [Φ]

0.000355 g of the compound 32, 0.000455 g of the compound 33, and 0.000557 g of N-methylacridine used as a quantum yield standard were weighted. Each sample was dissolved in 1 mL of pure methanol and the resulting solutions were placed in 5 mL volumetric flasks. Then 4 mL methanol was added to each of the volumetric flasks. For each solution, UV-Vis spectrum was measured using cuvettes with an optical path length of 1 cm. The maximum absorbance for each sample did not exceed 0.1 to ascertain a linear dependence of the fluorescence intensity and concentration. The measured samples and the standard were excited at identical conditions and absorbance values at the wavelength used for excitation, the same for the compound 32 or 33 and the standard, were read. For all these samples, emission spectra were recorded. The fluorescence quantum yields Φ were calculated according to the equation:

$$\frac{{S}_{x}}{{S}_{R}} \times \frac{{A}_{R}^{λ}}{{A}_{x}^{λ}} \times \frac{{n}_{x}^{2}}{{n}_{R}^{2}} \times {Φ}^{R}$$

wherein: x—measured substance; R—reference compound; S—area under the emission curve; A—absorbance at the same wavelength chosen for the excitation of x and R; n—refractive index of the solvent in which x and R are investigated; the term

$$\frac{{n}_{x}^{2}}{{n}_{R}^{2}}$$

represents a correction for different polarity of the solvents used, Φ—quantum yield of the reference compound. The results are listed in Table 1 in the third column.

TABLE 1

| | molar absorption coefficient | fluorescence quantum yield |
|---|---|---|
| | ε [M−¹ cm⁻¹]* | Φ_{Fl} ** |
| 2 | 13800 (357 nm) | 0.21 |
| | 66800 (292 nm) | |
| | 53500 (221 nm) | |
| 3 | 29700 (370 nm) | 0.25 |
| | 65500 (270 nm) | |

*Molar absorption coefficient ε [dm³ mol⁻¹ cm⁻¹] or [M⁻¹ cm⁻¹], samples weighted with an accuracy of 0.00002 g, 3 repetitions
** Fluorescence quantum yield Φ_{Fl} (determined relative to 10-methylacridine, Φ_{Fl} = 0.88 used as reference)

The compounds of the invention are applied as thermosensitive fluorescent labels for labeling of biological material with fluorescent dyes used for biological, chemical or physical analyzes.

These compounds are applied as fluorescent labels, especially for labeling nucleic acids, nucleosides, nucleotides, peptides or proteins, which can be detached from the labeled biomolecules without damaging or degrading it.

The examples provide exemplary procedures of biomolecule labeling, procedures of the label removal, and examples of determining the conditions suitable for the label removal. In the figures, selected results are presented that illustrate the curves of the marker removal versus time and temperature, the fluorescence intensity differences between the labeled compounds and the cyclic species resulting from the dye removal, and HPLC analyses illustrating this process.

### EXAMPLE 1

### Conversion of 2-(pyridine-2-yl)vinyl]pyridine Amino Alcohols into Imidazole-Carbonyl Esters

(E)-2-(methyl{6-[2-(pyridin-2-yl)vinyl]pyridin-2-yl}amino) ethan-1-ol (1 eq., 0.1 mmol) was dissolved in anhydrous acetonitrile (2 ml) and placed in a flask containing a magnetic stir bar. To this solution, 1′1′-carbonyldiimidazole (2 eq., 0.24 mmol) was added and the reaction was carried out for 30 minutes at room temperature and its course was monitored by TLC (ethyl acetate/n-hexane 9/1). After 30 minutes, the active form of imidazole-carbonyl ester was obtained, which was further used in the next example without any isolation

### EXAMPLE 2

### Labeling of 3′-O-Acetylthymidine with an Imidazole-Carbonyl Form of a Fluorescent Dye

To the solution of the imidazole-carbonylderivative obtained in the previous example 3′-O-acetylthymidine (1 eq; 0.1 mmol) and 1,1,3,3-tetramethylguanidine (1.6 eq., 0.16 mmol) were added. The reaction mixture was stirred for 2 hours at room temperature. The course of the reaction was monitored by TLC (dichloromethane/methanol 95/5 v/v). The solvent was evaporated under reduced pressure and the thus obtained residue was purified by silica gel column chromatography (dichloromethane (DCM)/MeOH; 0-5% MeOH) to obtain a pure product which was characterized by spectroscopic methods.

¹H NMR (400 MHz; Chloroform-d) δ 8.66-8.55 (m; 1H); 7.70-7.65 (m; 1H); 7.63 (d; J=5.3 Hz; 1H); 7.48 (d; J=8.6 Hz; 1H); 7.44 (d; J=8.0 Hz; 2H); 7.35 (d; J=2.2 Hz; 2H); 7.14 (dd; J=7.5; 4.9 Hz; 1H); 6.71 (d; J=7.3 Hz; 1H); 6.45 (d; J=8.5 Hz; 1H); 6.35 (dd; J=8.8; 5.7 Hz; 1H); 5.16 (dd; J=5.2; 3.2 Hz; 1H); 4.52 (dt; J=11.5; 5.9 Hz; 1H); 4.42 (q; J=4.6; 3.7 Hz; 1H); 4.39 (d; J=2.9 Hz; 1H); 4.33 (dd; J=11.8; 2.8 Hz; 1H); 4.17 (q; J=2.7 Hz; 1H); 4.02 (tq; J=14.7; 8.8; 7.4 Hz; 2H); 3.10 (s; 3H); 2.40-2.29 (m; 1H); 2.22-2.10 (m; 2H); 2.09 (s; 3H); 1.88 (s; 3H),

¹³C NMR (101 MHz; Chloroform-d) δ 169.89; 163.00; 157.25; 155.08; 154.11; 152.11; 149.93; 149.18; 137.44; 136.01; 134.34; 132.11; 130.24; 127.82; 122.10; 121.78; 112.34; 111.15; 104.99; 84.04; 81.62; 74.07; 66.76; 65.86; 47.87; 36.73; 36.64; 20.39; 12.13.

### EXAMPLE 3

### Conversion of 2-(pyridine-2-yl)vinyl]pyridinyl-aminoalcohols into [1,2,4]-triazole-carbonyl Esters

(E)-2-(methyl{6-[2-(pyridin-2-yl)vinyl]pyridin-2-yl}amino) ethan-1-ol (1 eq., 0.1 mmol) was dissolved in anhydrous acetonitrile (2 ml) and placed in a flask containing a magnetic stir bar. To this solution, 1′1′-carbonyldi[1,2,4]triazole (2 eq., 0.24 mmol) was added and the reaction was carried out for 10 minutes at room temperature and its course was monitored by TLC (ethyl acetate/n-hexane 9/1). After 10 minutes, active [1,2,4] triazole-carbonyl ester was obtained, which was further used in the next example without any isolation

### EXAMPLE 4

### Labeling of 3′-O-acetylthymidine with [1,2,4] triazole-carbonyl Ester of a Fluorescent Dye

To the solution of [1,2,4]triazole-carbonyl ester obtained in the previous example 3′-O-acetylthymidine (1 eq; 0.1 mmol) was added. The reaction mixture was stirred for 0.5 hours at room temperature. The course of the reaction was monitored by TLC (dichloromethane/methanol 95/5 v/v). The solvent was evaporated under reduced pressure and the thus obtained residue was purified by silica gel column chromatography (dichloromethane (DCM)/MeOH; 0-5% MeOH) to obtain a pure product which was characterized by spectroscopic methods. Because the product obtained is the same compound as in Example 2, the spectroscopic characteristics are given above in Example 2.

### EXAMPLE 5

### Label Detachment from 3′-O-acetyl-5′-(2-{methyl-[6-(2-pyridin-2-yl-vinyl)-pyridin-2-yl)-aminopropyloxycarbonylthymidine

The labeled nucleoside obtained in Example 2 was dissolved in phosphate buffer pH 6.86, in 5 portions of 1 mg each, and placed in sealable vials. Then, each sample was heated under shaking to the following temperatures: 30, 40, 50, 60 and 70° C. for the same period of time of 2 hours. Then the samples were quickly cooled down (in dry ice), from each sample an aliquot was taken and analyzed using a HPLC setup equipped with a DAD detector (diode array detector for the spectral range of 220-700 nm) and FLD detector (λ_{abs}=370 nm, λₜ=476 nm), using a Synergy Fusion—RP 80 Å column (together with Phenomenex pre-column), measuring 150×4.6 mm, using the gradient method: buffer A (0.01M triethylammonium acetate)/buffer B (0.01M triethylammonium acetate in 40% acetonitrile), flow rate 1 ml/min, analysis duration 30 min. The results of the kinetics of the label removal are presented on the graph: decay rate of the labeled species in function of temperature—FIG. 5. Complete removal of the label occurs at 70° C.

### EXAMPLE 6

(E)-2-(methyl{6-[2-(pyridin-2-yl)vinyl]pyridin-2-yl}amino)propan-1-ol (1 eq., 0.13 mmol, 35 mg) was dissolved in anhydrous acetonitrile (2 ml) and placed in a flask containing a magnetic stir bar together with 1′1′-carbonyldiimidazole (1 eq., 0.13 mmol, 38.09 mg). The reaction was carried out for 30 minutes at room temperature and was monitored by TLC (ethyl acetate/n-hexane 9/1). After 30 minutes, the active carbamate form of (E)-2-(methyl{6-[2-(pyridin-2-yl) vinyl] pyridin-2-yl} amino) propan-1-ol was obtained and used in the following example without any isolation.

### EXAMPLE 7

To the solution of the carbamate derivative of (E)-2-(methyl{6-[2-(pyridin-2-yl) vinyl] pyridin-2-yl} amino)propan-1-ol obtained in the previous example 6 3′-O-acetyl-thymidine (1 eq.; 0.13 mmol, 38.09 mg) and 1,1,3,3-tetramethylguanidine (1.6 eq., 0.21 mmol, 20 uL) were added. The reaction mixture was stirred for 2 hours at room temperature. The course of the reaction was monitored by TLC (DCM/methanol 95/5 v/v). The solvent was evaporated under reduced pressure and the thus obtained residue was purified by silica gel column chromatography (DCM/MeOH; 0-5% MeOH) to obtain a pure product (19 mg) which was characterized by spectroscopic methods.

¹H NMR (500 MHz, Chloroform-d) δ 8.67-8.57 (d, J=4.01 1H); 7.70-7.65 (m, 1H); 7.63 (d, J=5.3 Hz, 1H); 7.48 (d, J=8.6 Hz, 1H); 7.44 (d, J=8.0 Hz, 2H); 7.35 (d, J=2.2 Hz, 2H); 7.14 (dd, J=7.5, 4.9 Hz, 1H); 6.71 (d, J=7.3 Hz, 1H); 6.35 (d, J=8.5 Hz, 1H); 6.35 (dd, J=8.8, 5.7 Hz, 1H); 5.16 (dd, J=5.2, 3.2 Hz, 1H); 4.52 (dt, J=11.5, 5.9 Hz, 1H); 4.42 (q, J=4.6, 3.7 Hz, 1H); 4.39 (d, J=2.9 Hz, 1H); 4.33 (dd, J=11.8, 2.8 Hz, 1H); 4.17 (q, J=2.7 Hz, 1H); 4.02 (tq, J=14.7, 8.8, 7.4 Hz, 2H); 3.10 (s, 3H); 2.43-2.39 (m, 1H); 2.24-2.19 (m, 2H); 2.15-2.08 (m, 2H); 2.04 (s, 3H); 1.82 (s, 3H).

¹³C NMR (101 MHz, Chloroform-d) δ 170.40; 163.22; 157.86; 155.44; 154.64; 152.11; 149.93; 149.21; 137.78; 136.82; 134.87; 132.11; 130.24; 128.30; 122.10; 121.78; 112.57; 111.63; 105.60; 84.52; 81.62; 74.07; 66.76; 65.86; 47.87; 36.73; 36.64; 25.16; 20.39; 12.58.

### EXAMPLE 8

### Label Detachment from 3′-O-acetyl-5′-(2-{methyl-[6-(2-pyridin-2-yl-vinyl)-pyridin-2-yl)-aminopropyloxycarbonylthymidine

The labeled nucleoside obtained in Example 5 (1 mg) was dissolved in phosphate buffer pH 6.86 (0.5 mL, pH=7). The sample was heated (90° C.) and then in time intervals of 1 hour 50 μm (0.05 μL) aliquots were taken and quickly cooled to 0° C. After collecting 4 aliquots, TLC analysis showing the progress of the detachment reaction was performed. The heating was carried out for 24 hours after which a complete decay of the starting compound was determined. The reaction product was isolated from the TLC plates. A mass analysis revealed a molecular mass that corresponds to the bicyclic six-membered product 15.

MS-ESI: C₁₆H₁₈N₃⁺ calculated 252.1495 [M]⁺, observed 252 [M]⁺;

### EXAMPLE 9

### Label Detachment from 3′-O-acetyl-5′-(2-{methyl-[6-(2-pyridin-2-yl-vinyl)-pyridin-2-yl)-aminopropyloxycarbonylthymidine

The labeled nucleoside obtained in Example 2 was dissolved in phosphate buffer pH 6.86 in a glass vial with a screw cap. Then, the sample was continuously shaken and heated for 45 minutes at 90° C. for 45 minutes. After 5, 15, 30, and 45 minutes, aliquots were taken from the reacting mixture. The aliquots were quickly cooled down and analyzed using a HPLC setup equipped with a DAD detector (diode array detector for the spectral range of 220-700 nm) and FLD detector (λ_{abs.}=370 nm, λₜ=476 nm), using a Synergy Fusion—RP 80 Å column (together with a Phenomenex pre-column), measuring 150×4.6 mm, using the gradient method: buffer A (0.01M triethylammonium acetate)/buffer B (0.01M triethylammonium acetate in 40% acetonitrile), flow rate 1 ml/min, analysis duration 30 min. The results of the kinetics of the label removal are presented on the graph: the decay rate of the labeled species plotted versus temperature—FIG. 6. Complete removal of the label occurs after 45 minutes.

### EXAMPLE 10

### Formation of a Cyclic Dye Species as a Result of the Herein Developed Label Detachment Method from 3′-O-acetyl-5′-(2-{methyl-[6-(2-pyridin-2-yl-vinyl)-pyridin-2-yl)-aminoethyloxycarbonylthymidine

The labeled nucleoside obtained in Example 2 was dissolved in phosphate buffer pH 6.86 in a glass vial with a screw cap. The sample was continuously shaken and heated for 45 minutes at 90° C. for 45 minutes until complete decay of the labeled biomolecule which was determined by means of a thin layer plate chromatography (TLC). The solvent was evaporated under reduced pressure and the thus obtained residue was purified by column chromatography on a silica gel column.

¹H NMR (400 MHz, DMSO-d₆) δ 8.68 (dd, J=5.1, 1.7 Hz, 1H), 8.04 (dd, J=8.8, 7.6 Hz, 1H), 7.91 (td, J=7.7, 1.8 Hz, 1H), 7.82 (d, J=15.7 Hz, 1H), 7.72 (dt, J=7.9, 1.1 Hz, 1H), 7.59 (d, J=15.7 Hz, 1H), 7.48-7.44 (m, 1H), 7.43-7.39 (m, 1H), 7.12 (d, J=8.8 Hz, 1H), 4.80 (dd, J=10.6, 9.0 Hz, 2H), 3.95 (dd, J=10.6, 9.1 Hz, 2H), 3.10 (s, 3H), ¹³C NMR (101 MHz, DMSO-d₆) δ 172.75; 171.48; 154.94; 152.61; 150.02; 145.44; 144.12; 138.44; 137.39; 124.78; 120.41; 109.65; 106.01; 49.21; 48.09.

HRMS-ESI: C₁₅H₁₆N₃ calculated 238.1344 [M+H]⁺. found 238.1343 [M+H]⁺;

### EXAMPLE 11

### Conversion of (E)-2-(methyl{6-[2-(pyridin-2-yl)vinyl]pyridin-2-yl}amino)ethan-1-ol into Carbamate

(E)-2-(methyl{6-[2-(pyridin-2-yl)vinyl]pyridin-2-yl}amino)ethan-1-ol (1 eq., 25 mg, 0.098 mmol) was dissolved in anhydrous acetonitrile (2 ml) and placed in a flask containing a magnetic stir bar. Three portions of 1′1′-carbonyldiimidazole were added to the solution three times; each portion in an amount of 1.2 eq.; at 15 minute intervals (in total 3.6 eq; 57.10 mg; 0.36 mmol) and the reaction was carried out for a total of 1.5 hours at room temperature and its course was monitored by TLC (ethyl acetate/n-hexane 9/1). After 1.5 hours, the carbamate derivative of the aminoalcohol was obtained, which was further used in the next Example without any isolation.

### EXAMPLE 12

### Labeling of 3′-O-acetyl-N2-isobutyrylodeoxyguanosine with a Carbamate Derivative of a Fluorescent Dye

To the solution of carbamate aminoalcohol derivative obtained in Example 11 3′-O-acetyl-N2-isobutyrylodeoxyguanosine (1 eq.; 0.1 mmol) and 1,1,3,3-tetramethylguanidine (7.8 eq., 0.78 mmol) were added. The reaction mixture was stirred for 2 hours at room temperature. The course of the reaction was monitored by TLC (DCM/methanol 95/5 v/v). The solvent was evaporated under reduced pressure and the thus obtained residue was purified by silica gel column chromatography (DCM/MeOH; 0-5% MeOH) to obtain pure product with a yield of 30%.

### EXAMPLE 13

### Label detachment from 5′-[6-(2-pyridin-2-yl-vinyl)-pyridin-2-yl)-aminoethyloxycarbonyl N2-isobutyrylguanosine

The labeled N2-isobutyrylguanosine obtained according to the method described in Example 12 was dissolved in phosphate buffer pH 6.86, in 5 portions of 1 mg each, and placed in sealed vials. Then, each vial was heated under shaking to the following temperatures: 30, 40, 50, 60 and 70° C. for the same period of time of 2 hours. Then the samples were quickly cooled down (in dry ice) and analyzed using a HPLC setup equipped with a DAD detector (diode array detector for the spectral range of 220-700 nm) and FLD detector (λ_{abs}=370 nm, λₙ=476 nm), using a Synergy Fusion—RP 80 Å column (together with Phenomenex pre-column), measuring 150×4.6 mm, using the gradient method: buffer A (0.01 M triethylammonium acetate)/buffer B (0.01 M triethylammonium acetate in 40% acetonitrile), flow rate 1 ml/min, analysis duration 30 min. The results of the kinetics of the label removal are presented on the graph: the decay rate of the labeled species plotted versus temperature—FIG. 7. Complete removal of the label occurs at 70° C.

### EXAMPLE 14

### Conversion of 2-(pyridine-2-yl)vinyl]pyridinyl-aminoalcohols into Phosphoramidates

3(E)-2-(methyl{6-[2-(pyridin-2-yl)vinyl]pyridin-2-yl}amino)ethan-1-ol (80 mg; 0.313 mmol) was freeze-dried from benzene and dried under vacuum generated by an oil pump for 12 hours. The compound was then placed in a small round bottom flask flushed with argon and sealed with a septum. 1 ml of anhydrous dichloromethane was added to the flask to obtain a clear yellow solution. 40.9 mL (0.235 mmol) of N,N-diisopropylethylamine (DIPEA) was added in one portion and then the solution was cooled to about −5° C. To this clear solution, 41.75 mg (0.156 mmol) of N,N,N′,N′-bis(diisopropylamino)chlorophosphine was added. At the moment when a TLC analysis done in the developing system: hexane/DCM/triethylamine; v/v/v/; 6/3/1 revealed a half decay of the staring compound, 15 mg (0.078 mmol) of 5-benzylmercaptotetrazole (BMT) was added until complete disappearance of the starting compound. The solution was evaporated on a rotary evaporator and the product was purified by column chromatography (silica gel) applying a gradient elution in the system: DCM/hexane/triethylamine; v/v/v; from 1/8/1 to 2/7/1. Fractions containing the product were combined and evaporated on a rotary evaporator and then freeze-dried from benzene. Thus, 30 mg of bis[2-(pyridin-2-yl)vinyl]pirydynolo]-N,N-diisopropylphosphoramidite was obtained as yellow oil; with a yield of 30%.

The product was characterized by ³¹P NMR spectroscopy

³¹P NMR (400 MHz; benzene): 146.46 (s).

### EXAMPLE 15

### Conversion of 2-(pyridine-2-yl)vinyl]pyridinyl-aminoalcohols into Phosphoramidates

(E)-2-(methyl{6-[2-(pyridin-2-yl)vinyl]pyridin-2-yl}amino)ethan-1-ol (80 mg; 0.313 mmol) was freeze-dried from benzene and dried under vacuum generated by an oil pump for 12 hours. This compound was then placed in a small round bottom flask flushed with argon and sealed with a rubber cap. 1 ml of anhydrous dichloromethane was added to the flask to obtain a clear colorless solution. Then, 58.66 mL (1.3 eq; 0.177 mmol) of 2-cyanoethyl-N,N,N′,N′-bis(diisopropylamino)phosphine was added in one portion and the resulting solution was left for 5 minutes. Into this clear solution 5-benzyl-mercaptotetrazole (BMT) was added in portions of approximately 4.8 mg for one hour (in total 0.9 eq; 0.128 mmol, 23.7 mg). The resulting solution was left at room temperature for 1 hour. Then 70 μL of N,N-diisopropylamine (DIPA) was added to the solution in one portion and the whole was evaporated on a rotary evaporator. The product was purified by column chromatography (silica gel) using a gradient elution in the system DCM:hexane:triethylamine; v:v:v; 1:8:1 to 4:5:1. Fractions containing the product were combined and evaporated on a rotary evaporator; the resulting residue was immediately freeze-dried from benzene. Thus, approx. 25 mg of 2-(pyridin-2-yl)vinyl]pyridinyl-2-cyanoethyl-N,N-diizopropylphosphoramidite was obtained as fine powder.

The product was characterized by ³¹P NMR spectroscopy

³¹P NMR (400 MHz; benzene-d6): 146.46 (s)

### EXAMPLE 16

### Oligomer Labeling

A 12-mer oligomer with the sequence 5′-TTT TTT TTT TTT TTT-3′ was synthesized on a solid phase using a Q-dT silicate substrate (Glen Research) and commercial thymidine phosphoramidite. The synthesis was carried out using an automated DNA synthesizer. For each nucleotide being attached, 4 reaction steps (detritylation, condensation, capping, and oxidation) were done repeatedly. For this purpose, the Q-dT fixed-bed columns were filled with respective solutions using for each step the following conditions:
1 detritylation: 3% solution of dichloroacetic acid in methylene chloride, reaction time 2 minutes
2 condensation: 0.1 molar solution of dT phosphoramidite in acetonitrile mixed in equal proportions with a 0.1 molar solution of 5-benzyl-mercaptotetrazole (BMT), reaction time 3 minutes
3. capping: a mixture of two solutions (A and B) in equal proportions, reaction time 1 minute. Solution A—10% solution of 1-N-methylimidazole in tetrahydrofuran. Solution B—10% solution of acetic anhydride in anhydrous tetrahydrofuran.
4. oxidation: 0.05 molar solution of iodine in aqueous pyridine, reaction time 1 minute.

After each such step, the column is rinsed with anhydrous acetonitrile and flushed with dry argon.

After attachment of the last oligonucleotide, the final dimethoxytrityl group was removed (detritylation) and the condensation step was carried out using a mixture of a dye phosphoramidite derivative solution (phosphoramidite derivative of the dye (12 mg) dissolved in 200 μl of dry ACN) and 0.1 molar BMT solution in equal proportions. The reaction time was 10 minutes. Subsequently, the oxidation step was carried out as described above.

After completion of the oxidation step, the column was washed with acetonitrile, flushed with dry argon, and the medium bed was placed into a concentrated ammonia solution at 22° C. for 2 minutes. The silicate support was then removed from the solution by decantation and the labeled oligomer in ammonia solution was applied at the top of a LGC MicroPure II column. Then the column was eluted with the following mixtures with each fraction being collected into separate tubes:
1. 97% 1M triethylammonium acetate (TEAA)+3% MeCN v/v
2. ultra-pure water with a conductivity of 18.2 MΩ·cm at 25° C. (i.e. H₂O MiliQ)
3. 20% solution of acetonitrile in MiliQ water

The labeled oligonucleotide was contained in the fraction eluted with H₂O MiliQ as found spectrophotometrically (in an amount corresponding to 14 OD optical units). The oligonucleotide was evaporated without heating and was subjected to further analysis.

### EXAMPLE 17

### Purification of the Labeled Oligomer by HPLC

Preparation of the analyte: labeled oligonucleotide (0.4 OD) was dissolved in 500 mL of MiliQ water, mixed on a Vortex and centrifuged. OD was measured at 260 nm to determine the injection volume per chromatographic column. (OD260—0.528, Analyte volume—100 μl).

### Conditions of the Analysis:

A Synergy Fusion column—RP 80 Å Phenomenex, 150×4.6 mm with pre-column gradient method, duration 30 min:
flow rate 1 ml/min
gradient analysis using a buffer A (0.01M triethylammonium acetate)/buffer B (0.01M triethylammonium acetate in 40% acetonitrile) system
DAD diode detector 220-700 nm
FLD detector (λ_{abs.}=370 nm, λₑₘ=476 nm)

The labeled oligomer 1, purified by high pressure HPLC chromatography under specified above conditions, was evaporated and then re-evaporated 3 times from MiliQ water (in order to completely remove the remaining triethylammonium acetate buffer) and freeze-dried. The resulting analyte was analyzed by mass spectrometry (MALDI).

Calculated mass of the labeled oligonucleotide: 3904.6258 g/mol.

Found mass of the labeled oligonucleotide: m/z 3905.561 Da.

### EXAMPLE 18

Detachment of the Label Dye from the Oligomer

To remove the label from the oligonucleotide, the labeled oligonucleotide (in the amount of 10 OD) was dissolved in 1000 mL of MiliQ water, mixed on a Vortex and centrifuged.

Subsequently, OD was measured at 260 nm and the injection volume containing 6 OD of the oligomer was determined. The solution was placed in a screw cap vessel and heated for 4 hours at 90° C. After every hour, from the mixture a sample containing about 1 OD of the oligomer was taken and, after cooling (dry ice), each of them was subjected to an analysis with an HPLC set equipped with a DAD detector (diode array detector for the range 220-700 nm) and a FLD detector (λ_{abs.}=370 nm, λₜ=476 nm) using a Synergy Fusion—RP 80 Å column (together with a Phenomenex pre-column) measuring 150×4.6 mm, and using the gradient method: buffer A (0.01M triethylammonium acetate)/buffer B (0.01 M triethylammonium acetate in 40% acetonitrile), flow rate 1 ml/min, analysis time 30 min. The results of the dye removal analyzes are shown on HPLC chromatograms.

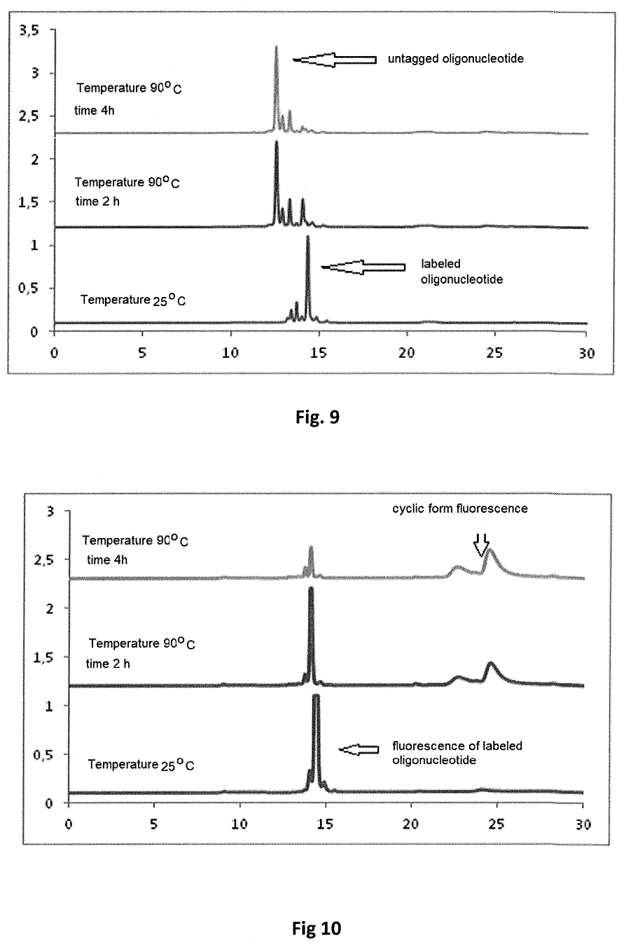

In the following:
FIG. 9 shows chromatograms of unheated oligomer and oligomer heated oligomer for 2 and 4 hours, respectively, that were recorded using DAD detector,
FIG. 10 shows chromatograms of unheated oligomer and the oligomer heated for 2 and 4 hours, respectively, recorded using Fluorescence detector at the excitation wavelength 370 and emission wavelength 480 nm.

TABLE 1
Retention time of individual compounds on the Synergy Fusion -
RP 80 Å column during analyses using the HPLC kit performed
under the conditions for the analyzed compounds as described above.

| Compound | Retention time [min] |
|---|---|
| Labeled oligonucleotide | 14.49 |
| Dye-free oligonucleotide | 12.57 |
| Cyclic derivative of the dye | 22.64-24.52 |

### EXAMPLE 19

### Preparation of (E)-{6-[2-(pyridin-2-yl)vinyl]pyridin-2-yl}-aminoetan-1-ol

(E)-2-bromo-6-[2-(pyridin-2-yl)vinyl)pyridine (1.23 mmol, 320 mg) in 0.5 mL of anhydrous DMF, diisopropyl ethanolamine (1.8 mmol, 0.3 mL) and a large excess of ethanolamine (1.5 mL) were placed in a round bottom flask. The reaction was carried out in a microwave reactor (120° C., 300 W) for 3 hours. After completion of the reaction, DMF was evaporated and the row product was preliminarily purified by extraction from dichloromethane (DCM)/saturated aqueous solution of NaHCO₃ (1/1; v/v). The product was then purified by silica gel column chromatography using DCM/methanol mixtures as eluents (gradient method, starting with one-component eluent (100% DCM) and ending with the mixture 95/5 v/v as the final eluent), and the solvents were evaporated on a rotary evaporator. To completely remove traces of the organic solvents the purified product was freeze-dried from benzene. Thus, 175 mg of (E)-{6-[2-(pyridin-2-yl)vinyl]-pyridin-2-yl}-aminoethan-1-ol was obtained as yellowish oil, with a yield of 60%.

The product was characterized by NMR spectroscopy:

¹H NMR (500 MHz, Chloroform-d) δ 8.48 (dd, J=5.0, 1.8 Hz, 1H. H6), 7.52 (dd, J=7.7, 1.8 Hz, 1H, H4), 7.48 (d, J=15.7 Hz, 1H, H7), 7.39 (d, J=15.7 Hz, 1H, H8), 7.32 (d, J==7.8 Hz, 1H, H3), 7.21 (dd, J=8.3, 7.2 Hz, 1H, H11), 7.06-6.99 (m, 1H, H5), 6.58 (d, J=7.2 Hz, 1H, H10), 6.23 (d, J=8.3 Hz, 1H, H12), 5.55 (s, 1H, H17 (—OH)), 5.32 (t, J=5.7 Hz, 1H, H14 (—NH)), 3.74 (t, J=4.8 Hz, 2H, H16), 3.45 (q, J=5.1 Hz, 2H, H15)

¹³C NMR (126 MHz, Chloroform-d) δ 158.47 (C13), 155.05 (C2), 152.11 (C9), 149.28 (C6), 137.63 (C11), 136.44 (C4), 131.87 (C8), 130.28 (C7), 122.70 (C3), 122.20 (C5)), 113.39 (C10), 108.50 (C12), 63.26 (C16), 44.71 (C15)

ESI MS m/z; calculated: 242.1293 C₁₄H₁₅N₃O [M+H]⁺; found: 242.1277 [M+H]⁺;

### EXAMPLE 20

### Preparation of (E)-2-(methyl-{6-[2-(pyridin-2-yl)vinyl]pyridin-2-yl}-amino)ethan-1-ol

(E)-2-bromo-6-[2-(pyridin-2-yl)vinyl)pyridine (0.61 mmol, 160 mg) in 0.5 mL of anhydrous DMF, diisopropyl ethanolamine (0.92 mmol, 159 μL), and a large excess of N-methylethanolamine (1.5 mL) were placed in a round bottom flask. The reaction was carried out in a microwave reactor (110° C., 300 W). After completion of the reaction, DMF was evaporated and the row product was preliminarily purified by extraction from DCM/water/saturated aqueous solution of NaHCO₃ (1/1/1; v/v/v). The product was then purified by silica gel column chromatography using DCM/methanol mixtures as eluents (gradient method, starting with one-component eluent (100% DCM) and ending with the mixture 95/5 v/v as the final eluent), and the solvents were evaporated on a rotary evaporator. To completely remove traces of the organic solvents, the purified product was freeze-dried from benzene. Thus, 131 mg of (E)-2-(methyl-{6-[2-(pyridin-2-yl)vinyl]pyridin-2-yl}-amino)ethan-1-ol was obtained as yellowish oil, with a yield of 70%. The product was characterized by NMR spectroscopy:

¹H NMR (500 MHz, DMSO-d₆) δ 8.57 (dd, J=4.9, 1.7 Hz, 1H); 7.78 (td, J=7.7, 1.9 Hz, 1H); 7.60 (m, 1H); 7.56 (m, 1H); 7.52 (d, J=9.1 Hz, 1H); 7.50-7.46 (m, 1H); 7.26 (ddd, J=7.4, 4.7, 1.0 Hz, 1H); 6.75 (d, J=7.1 Hz, 1H); 6.59 (d, J=8.5 Hz, 1H); 4.73 (t, J=4.8 Hz, 1H); 3.63 (m, 4H); 3.10 (s, 3H)

¹³C NMR (126 MHz, DMSO-d₆) δ 157.77; 154.83; 151.88; 149.51; 137.69; 136.77; 132.49; 130.20; 122.55; 122.48; 111.65; 105.93; 58.58; 51.88; 36.73

### EXAMPLE 21

### Preparation of (E)-2-(benzyl{6-[2-(pyridin-2-yl)vinyl]pyridin-2-yl}amino)ethan-1-ol

(E)-2-bromo-6-[2-(pyridin-2-yl)vinyl)pyridine (0.76 mmol, 200 mg) in 0.5 mL DMF, diisopropylethanolamine (1.14 mmol, 0.19 mL), and a large excess of 2-(benzylamino)ethanol (1.5 mL) were placed in a round bottom flask. The reaction was carried out in a microwave reactor (120° C., 300 W) for 5 hours. After completion of the reaction, DMF was evaporated and the row product was preliminarily purified by extraction from dichloromethane (DCM)/saturated aqueous solution of NaHCO₃ (1/1; v/v). The product was then purified by silica gel column chromatography using hexane/ethyl acetate mixtures as eluents (gradient method, starting with one-component eluent (100% hexane) and ending with the mixture 1/1 v/v as the final eluent), and the solvents were evaporated on a rotary evaporator. To completely remove traces of the organic solvents, the purified product was freeze-dried from benzene. Thus, 87 mg of (E)-2-(benzyl{6-[2-(pyridin-2-yl)vinyl]pyridin-2-yl}amino)ethan-1-ol was obtained as yellowish oil, with a yield of 34%. The product was characterized by NMR spectroscopy.

### EXAMPLE 22

### Synthesis of (E)-2-(methyl-{6-[2-(pyridin-2-yl)vinyl]pyridin-2-yl]amino)-1-phenylethane-1-ol

100 mg (1 eq., 0.383 mmol) of (E)-2-bromo-6-[2-(pyridin-2-yl)vinyl)pyridine and 289.6 mg (5 eq., 1.915 mmol) of α-(methylamino-methyl) benzyl alcohol were transferred into a high pressure tube and dissolved in 3 ml of anhydrous N-methylpyrrolidone (NMP). Then, 667 μl (10 eq., 3.83 mmol) of N,N-diisopropylethylamine (DIPEA) dried over molecular sieves was added in a single portion. The tube was sealed with a rubber septum and purged with argon. The reaction was carried out in a microwave reactor for 2 h at given operation parameters: Pₘₐₓ=100 W, Tₘₐₓ=160° C.). After completion of the reaction, the solvent was evaporated on a rotary evaporator and the residue was dissolved in a small amount of dichloromethane and applied directly to a silica gel column. The reaction mixture was purified by column chromatography, by eluting with an isocratic system: CH₂Cl₂/CH₃OH (99/1 v/v). The main product fractions were combined, evaporated with a rotary evaporator, and freeze-dried from benzene to remove residual organic solvents. 70 mg of (E)-2-(methyl-{6-[2-(pyridin-2-yl)vinyl]pyridin-2-yl]amino)-1-phenylethan-1-ol (yield=55%) as yellow oil.

¹H NMR (400 MHz; Chloroform-d) δ 2.87 (s; 3H); 3.91 (m; 2H); 5.13 (dd; J=2.7; 6.7 Hz; 1H; CH); 6.46 (d; J=8.5 Hz; 1H); 6.79 (d; J=7.2 Hz; 1H); 7.17 (ddd; J=0.6; 4.9; 7.3 Hz); 7.28 (t; J=7.3 Hz); 7.37 (t; J=7.3 Hz; 2H); 7.45-7.51 (m; 4H); 7.57 (d; J=15.7 Hz; 1H; HC═); 7.67 (d; J=15.7 Hz; 1H); 7.67 (d; J=1.7 Hz; 1H); 8.62 (d; J=4.2 Hz; 1H).

¹³C NMR (101 MHz, Chloroform-d) δ 38.37; 60.17; 75.03; 106.12; 113.30; 122.48; 123.36; 125.97; 127.17; 128.26; 131.00; 131.72; 136.56; 138.48; 143.37; 149.68; 152.32; 155.17; 159.12.

ESI MS m/z found: 332.354; calculated for C₂₁H₂₂N30 [M+H]⁺ 332.4; C₂₁H₂₁N₃ON [M+Na]⁺ 354.4.

### EXAMPLE 23

### Synthesis of (E)-2-(methyl-{6-[2-(pyridin-2-yl)vinyl]pyridin-2-yl]amino)ethan-1-ol

110 mg (1 eq., 0.35 mmol) of (E)-2-bromo-6-[2-(pyridin-2-yl)-vinyl)pyridine and 140 μl (5 eq., 1.75 mmol) of 2-(methylamino)ethanol were transferred into a high pressure tube and dissolved in 3 ml of anhydrous N-methylpyrrolidone (NMP). Then, 610 μl (10 eq., 3.5 mmol) of N,N-diisopropylethylamine (DIPEA) dried over molecular sieves was added in a single portion. The tube was sealed with a rubber cap and purged with argon. The reaction was carried out in a microwave reactor for 2 h at given operation parameters: Pₘₐₓ=100 W, Tₘₐₓ=160° C.). After completion of the reaction, the solvent was evaporated on a rotary evaporator and the residue was dissolved in a small amount of dichloromethane and applied directly to a silica gel column. The reaction mixture was purified by column chromatography, by eluting with an isocratic system: CH₂Cl₂/CH₃OH (99/1 v/v). The main product fractions were combined, evaporated with a rotary evaporator, and freeze-dried from benzene to remove residual organic solvents. 73 mg of (E)-2-(methyl-{6-[2-(quinolin-2-yl)vinyl]-pyridin-2-yl}-amino)ethan-1-ol was thus obtained (yield=68%) as yellow viscous oil.

¹H NMR (400 MHz; Chloroform-d): 3.11 (s; 3H); 3.87 (t; J 4.9=Hz; 2H); 3.94 (t; J=5.0 Hz; 2H); 6.52 (d; J=8.5 Hz; 1H); 6.83 (d; J=7.2 Hz; 1H); 7.47-7.54 (m; 2H); 7.64 (d; J=15.9 Hz; 1H); 7.73 (td; J=8.2; 1.0 Hz; 1H); 7.79 (t; J=7.3 Hz; 2H); 8.20 (t; J=8.2 Hz; 2H).

### EXAMPLE 24

### Synthesis of (E)-2-(methyl-{6-[2-(3-methoxyphenyl)-vinyl]-pyridin-2-yl]amino)ethan-1-ol

(E)-2-bromo-6-[2-(3-methoxyphenyl)vinyl)pyridine (0.61 mmol, 176 mg) in 0.5 mL of anhydrous DMF, diisopropyl ethanolamine (0.92 mmol, 159 μL), and a large excess of N-methylethanolamine (1.5 mL) were placed in a round bottom flask. The reaction was carried out in a microwave reactor (110° C., 300 W). After completion of the reaction, DMF was evaporated and the row product was preliminarily purified by extraction from DCM/water/saturated aqueous solution of NaHCO₃ (1/1/1; v/v/v). The product was then purified by silica gel column chromatography using DCM/methanol mixtures as eluents (gradient method, starting with one-component eluent (100% DCM) and ending with the mixture 95/5 v/v as the final eluent), and the solvents were evaporated on a rotary evaporator. To completely remove traces of the organic solvents, the purified product was freeze-dried from benzene. Thus, 125 mg of (E)-2-(methyl-{6-[2-(3-methoxyphenyl)vinyl]pyridin-2-yl}-amino)ethan-1-ol was obtained as yellowish oil, with a yield of 72%.

### EXAMPLE 25

### Preparation of (E)-2-(methyl-{6-[2-(pyridin-2-yl)vinyl]pyridin-2-yl}-amino) propan-1-ol

(E)-2-bromo-6-[2-(pyridin-2-yl)vinyl)pyridine (0.61 mmol, 160 mg) in 0.5 mL of anhydrous DMF, diisopropyl ethanolamine (0.92 mmol, 159 μL), and a large excess of N-methylaminopropan-1-ol (1.2 mmol) were placed in a round bottom flask. The reaction was carried out in a microwave reactor (120° C., 300 W). After completion of the reaction, DMF was evaporated and the row product was preliminarily purified by extraction from DCM/water/saturated aqueous solution of NaHCO₃ (1/1/1; v/v/v). The product was then purified by silica gel column chromatography using DCM/methanol mixtures as eluents (gradient method, starting with one-component eluent (100% DCM) and ending with the mixture 97/3 v/v as the final eluent), and the solvents were evaporated on a rotary evaporator. To completely remove traces of the organic solvents, the purified product was freeze-dried from benzene. Thus, 125 ng of (E)-2-(methyl-{6-[2-(pyridin-2-yl)-vinyl]pyridin-2-yl}-amino)propan-1-ol was obtained as yellowish oil, with a yield of 67%.

¹H NMR (500 MHz, Chloroform-d) δ 8.53 (d, J=4.1 Hz, 1H); 7.57 (td, J=7.7, 1.8 Hz, 1H); 7.45-7.41 (m, 2H); 7.41-7.36 (m, 2H); 7.07 (ddd, J=7.6, 4.8, 1.1 Hz, 1H); 6.67 (d, J=7.2 Hz, 1H); 6.40 (d, J=8.5 Hz, 1H); 3.85-3.79 (m, 2H); 3.46 (t, J=5.5 Hz, 2H); 2.92 (s, 3H); 1.76 (p, J=5.6 Hz, 2H); 1.66 (s, 1H).

¹³C NMR (126 MHz, CDCl₃) δ 158.88; 155.41; 152.88; 149.77; 138.54; 136.60; 132.26; 131.06; 123.08; 122.53; 112.37; 105.44; 58.12; 45.24; 35.76; 30.20.

### EXAMPLE 26

### Synthesis of (E)-2-(methy-{6-[2-(quinolin-2-yl)vinyl]pyridin-2-yl]amino)propan-1-ol

110 mg (1 eq., 0.35 mmol) of (E)-2-bromo-6-[2-(quinolin-2-yl)-vinyl)pyridine and 140 μl (5 eq., 1.75 mmol) of 3-methylaminopropan-1-ol were transferred into a high pressure tube and dissolved in 3 ml of anhydrous N-methylpyrrolidone (NMP). Then, 610 μl (10 eq., 3.5 mmol) of N, N-diisopropylethylamine (DIPEA) dried over molecular sieves was added in a single portion. The tube was sealed with a rubber cap and purged with argon. The reaction was carried out in a microwave reactor for 2 h at given operation parameters: Pₘₐₓ=100 W, Tₘₐₓ=170° C.). After completion of the reaction, the solvent was evaporated on a rotary evaporator and the residue was dissolved in a small amount of dichloromethane and applied directly to a silica gel column. The reaction mixture was purified by column chromatography, by eluting with an isocratic system: CH₂Cl₂/CH₃OH (99/1 v/v). The main product fractions were combined, evaporated with a rotary evaporator, and freeze-dried from benzene to remove residual organic solvents. 65 mg of (E)-2-(methyl-{6-[2-(quinolin-2-yl)vinyl]-pyridin-2-yl}-amino)propan-1-ol was thus obtained (yield=57%) as yellow viscous oil.

### EXAMPLE 27

### Preparation of 2-((6-bromo-pyridin-2-yl)amino)-ethan-1-ol

2,6-dibromopyridine (0.03 mol; 6.5 g) in 10 mL of pyridine, ethanolamine (0.033 mol, 2 mL) and diisopropylethylamine (0.045 mol, 7.7 mL) were placed in a round bottom flask. The mixture was stirred using a magnetic stirrer and heated to 120° C. in a microwave reactor for 5 hours (300 W), controlling the reaction by means of a thin layer chromatography. After completion of the reaction, the solvents were evaporated and the product was extracted using dichloromethane (DCM)/saturated aqueous solution of NaHCO₃ (1/1 v/v). The product was then purified by silica gel column chromatography using hexane/ethyl acetate mixtures as eluents (gradient method, starting with one-component eluent (100% hexane) and ending with the mixture 7/3 v/v as the final eluent), and the solvents were evaporated on a rotary evaporator. To completely remove traces of the organic solvents, the purified product was freeze-dried from benzene. Thus, 4.4 g of 2-(6-bromo-(pyridin-2-yl)amino)-ethan-1-ol was obtained as colorless oil in a 73% yield. The product was characterized by NMR spectroscopy:

¹H NMR (400 MHz, Chloroform-d) δ 7.19 (dd, J=8.2, 7.5 Hz, 1H, H4); 6.68 (dd, J=7.5, 0.6 Hz, 1H, H5); 6.32 (dd, J=8.3; 0.6 Hz, 1H, H3); 5.24 (t, J=5.7 Hz, 1H, —NH); 3.98 (s, 1H, —OH); 3.78 (t, J=4.9 Hz, 2H, H8); 3.41 (td, J=5.6; 4.4 Hz, 2H, H7).

¹³C NMR (101 MHz, Chloroform-d) δ 158.96 (C2); 139.68 (C6); 139.50 (C4); 115.74 (C5); 105.77 (C3); 61.98 (C8); 44.59 (C7),

ESI MS m/z; calculated: 216.9976 C₇H₉BrN₂O [M+H]⁺; found: 238.9802 C₇H₉BrN₂ON [M+Na]⁺.

### EXAMPLE 28

### Preparation of 3-((6-bromo-pyridin-2-yl)amino)-propan-1-ol

2,6-dibromopyridine (0.03 mol; 6.5 g) in 10 mL of pyridine, propanolamine (0.033 mol, 2 mL) and diisopropylethylamine (0.045 mol, 7.7 mL) were placed in a round bottom flask. The mixture was stirred using a magnetic stirrer and heated to 120° C. in a microwave reactor for 5 hours (300 W), controlling the reaction by means of a thin layer chromatography. After completion of the reaction, the solvents were evaporated and the product was extracted using DCM/saturated aqueous solution of NaHCO₃ (1/1 v/v). The product was then purified by silica gel column chromatography using hexane/ethyl acetate mixtures as eluents (gradient method, starting with one-component eluent (100% hexane) and ending with the mixture 7/3 v/v as the final eluent), and the solvents were evaporated on a rotary evaporator. To completely remove traces of the organic solvents the purified product was freeze-dried from benzene. 3.8 g of 3-((6-bromo-pyridin-2-yl) amino)-propan-1-ol were obtained as colorless oil in a 59% yield.

### EXAMPLE 29

### Preparation of 2-[(2,4-difluorobenzyl)(6-bromo-pyridin-2-yl)amino]ethan-1-ol

2-((6-bromo-pyridin-2-yl)amino)ethan-1-ol (0.0046 mol, 1 g) in 5 mL DMF, 1-(bromomethyl)-2,4-difluorobenzene (0.0055 mol, 0.7 mL), and diisopropylethylamine (0.007 mol, 1.13 mL) were placed in a round bottom flask. The mixture was stirred using a magnetic stirrer and heated to 120° C. in an oil bath under reflux for 24 hours, controlling the reaction by means of a thin layer chromatography. After completion of the reaction, the solvents were evaporated and the product was extracted using DCM/saturated aqueous solution of NaHCO₃ (1/1 v/v). The product was then purified by silica gel column chromatography using hexane/ethyl acetate mixtures as eluents (gradient method, starting with one-component eluent (100% hexane) and ending with the mixture 7/3 v/v as the final eluent), and the solvents were evaporated on a rotary evaporator. To completely remove traces of the organic solvents, the purified product was freeze-dried from benzene. Thus, 950 mg of 2-[(2,4-difluorobenzyl)-(6-bromo-pyridin-2-yl) amino]-ethanol was obtained as oil in a 63% yield. The product was characterized by NMR spectroscopy:

¹H NMR (500 MHz, Chloroform-d) δ 7.30-7.26 (m, 1H, H4); 7.20 (td, J=8.7; 6.4 Hz, 1H, H13); 6.90-6.82 (m, 211, H10, 11); 6.79 (d, J=7.4 Hz, 1H, H5), 6.42 (d, J=8.4 Hz, 1H, H3); 4.71 (s, 2H, H9); 3.90 (dd, J=5.5; 4.5 Hz, 2H, H8); 3.80 (t, J=5.0 Hz, 2H, H7).

¹³C NMR (126 MHz, Chloroform-d) δ 162.51 (dd, J=194.7; 11.8 Hz, C14); 160.54 (dd, J=194.3; 11.9 Hz, C12); 158.54 (C2); 139.88 (C4); 139.37 (C6); 129.28 (dd, J=9.6; 5.9 Hz, C10); 119.96 (dd, J=14.9; 3.7 Hz, C15); 115.92 (C5); 111.45 (dd, J=21.1; 3.7 Hz, C11); 104.86 (C3); 104.61-103.48 (m, C13); 62.15 (C8); 52.28 (C7); 47.04 (C9),

ESI MS m/z; calculated: 343.0258 C₁₄H₁₃BRF₂N₂O [M+H]⁺; found: 343.0255 [M+H]⁺.

### EXAMPLE 30

### Preparation of 2-[(2,4-dimethylbenzyl)(6-bromo-pyridin-2-yl)amino]ethan-1-ol

2-((6-bromo-pyridin-2-yl)amino)ethan-1-ol (0.0029 mol; 0.63 g) in 5 mL DMF, 1-(bromomethyl)-2,4-dimethylbenzene (0.0035 mol; 0.53 mL), and diisopropylethylamine (0.0045 mol; 0.71 mL) were placed in a round bottom flask. The mixture was stirred using a magnetic stirrer and heated to 110° C. in an oil bath under reflux for 48 hours, controlling the reaction by means of a thin layer chromatography. After completion of the reaction, the solvents were evaporated and the product was extracted using DCM/saturated aqueous solution of NaHCO₃ (1/1 v/v). The product was then purified by silica gel column chromatography using hexane/ethyl acetate mixtures as eluents (gradient method, starting with one-component eluent (100% hexane) and ending with the mixture 7/3 v/v as the final eluent), and the solvents were evaporated on a rotary evaporator. To completely remove traces of the organic solvents, the purified product was freeze-dried from benzene. Thus, 560 mg of 2-[(2,4-dimethylbenzyl)(6-bromo-pyridin-2-yl)amino]ethan-1-ol was obtained as oil, with a yield of 58%. The product was characterized by NMR spectroscopy:

¹H NMR (400 MHz, Chloroform-d) S 7.20 (dd, J=8.4, 7.5 Hz, 1H, H4), 7.04 (s, 1H, H13), 6.96 (dd, J=7.9, 1.7 Hz, 1H, H10), 6.91 (d, J=7.8 Hz, 1H, H1), 6.74 (d, J=7.4 Hz, 1H, H5), 6.27 (d, J=8.4 Hz, 1H, H3), 4.55 (s, 2H, H9), 3.87 (td, J=4.7, 1.0 Hz, 2H, H8), 3.82-3.72 (m, 2H, H7), 2.31 (s, 3H, p-CH3), 2.28 (s, 3H, m-CH3). ¹³C NMR (101 MHz, Chloroform-d) δ 159.10 (C2), 139.75 (C4), 139.15 (C6), 136.87 (C14), 135.37 (C12), 131.49 (C13), 130.89 (C15), 126.82 (C10), 125.48 (C11), 115.43 (C5), 105.09 (C3), 62.81 (C8), 52.35 (C7), 51.46 (C9), 20.88 (C16), 18.88 (C17),

ESI MS m/z; calculated: 335.0759 C₆H₁₉BrN₂O [M+H]⁺; found: 335.0757 [M+H]⁺.

### EXAMPLE 31

### Preparation of 2-[(2-fluorobenzyl)(6-bromo-pyridin-2-yl)amino]ethan-1-ol

2-((6-bromo-pyridin-2-yl)amino)ethan-1-ol (0.0037 mol; 0.8 g) in 5 mL of DMF, 1-(bromomethyl)-2-fluorobenzene (0.0044 mol; 0.54 mL), and diisopropylethylamine (0.0055 mol; 0.90 mL) were placed in a round bottom flask. The mixture was stirred using a magnetic stirrer and heated to 130° C. in an oil bath under reflux for 48 hours, controlling the reaction by means of a thin layer chromatography. After completion of the reaction, the solvents were evaporated and the product was extracted using DCM/saturated aqueous solution of NaHCO₃ (1/1 v/v). The product was then purified by silica gel column chromatography using hexane/ethyl acetate mixtures as eluents (gradient method, starting with one-component eluent (100% hexane) and ending with the mixture 7/3 v/v as the final eluent), and the solvents were evaporated on a rotary evaporator. To completely remove traces of the organic solvents, the purified product was freeze-dried from benzene. Thus, 640 mg of 2-[(2-fluorobenzyl)(6-bromo-pyridin-2-yl)amino]ethan-1-ol was obtained as oil in a 54% yield. The product was characterized by NMR spectroscopy:

¹H NMR (500 MHz, Chloroform-d) δ 7.34-7.27 (m, 2H, H4, H14); 7.21 (td, J=7.7; 1.8 Hz, 1H, H12); 7.16-7.10 (m, 2H, H13, H11); 6.80 (d, J=7.4 Hz, 1H, H5); 6.43 (d, J=8.4 Hz, 1H, H3); 4.77 (s, 2H, H9); 3.92 (dd, J=5.5; 4.3 Hz, 2H, H8); 3.87-3.81 (m, 2H, H7); 3.39 (s, 1H, —OH),

¹³C NMR (126 MHz, Chloroform-d) δ 161.75 (d, J_{C10F}=250 Hz, C10); 159.80 (C2); 158.76 (C4); 139.75 (C6); 139.36 (C14); 128.95 (C12) (d, J=7.9 Hz); 128.11 (C13); 124.31 (C15) (d, J=3.6 Hz); 115.68 (C5); 115.45 (C11) (d, J=21.2 Hz); 104.83 (C3); 62.38 (C8); 52.38 (C7); 47.44 (C9) (d, J=4.8 Hz),

ESI MS m/z; calculated: 325.0352 C₁₄H₁₄BrFN₂O [M+H]⁺; found: 325.0349 [M+H]⁺.

### EXAMPLE 32

### Preparation of 2-[(2-chlorobenzyl)(6-bromo-pyridin-2-yl)amino]ethan-1-ol

2-((6-bromo-pyridin-2-yl)amino)ethan-1-ol (0.0037 mol; 0.8 g) in 5 mL of DMF, 1-(bromomethyl)-2-chlorobenzene (0.0043 mol; 0.58 mL), and diisopropylethylamine (0.0055 mol; 0.8 mL) were placed in a round bottom flask. The mixture was stirred using a magnetic stirrer and heated to 120° C. in an oil bath under reflux for 48 hours, controlling the reaction by means of a thin layer chromatography. After completion of the reaction, the solvents were evaporated and the product was extracted using DCM/saturated aqueous solution of NaHCO₃ (1/1 v/v). The product was then purified by silica gel column chromatography using hexane/ethyl acetate mixtures as eluents (gradient method, starting with one-component eluent (100% hexane) and ending with the mixture 7/3 v/v as the final eluent), and the solvents were evaporated on a rotary evaporator. To completely remove traces of the organic solvents, the purified product was freeze-dried from benzene. Thus, 460 mg of 2-[(2-chlorobenzyl)(6-bromo-pyridin-2-yl)amino]ethan-1-ol was obtained as oil with a yield of 57%. The product was characterized by NMR spectroscopy:

¹H NMR (500 MHz, Chloroform-d) δ 7.30-7.19 (m, 4H); 7.11 (dt, J=6.9, 1.9 Hz, 1H); 6.77 (d, J=7.4 Hz, 1H, H5); 6.36 (d, J=8.4 Hz, 1H, H3); 4.68 (s, 2H, H9); 3.88 (t, J=5.0 Hz, 2H, H8); 3.78 (t, J=5.0 Hz, 2H, H7),

¹³C NMR (126 MHz, Chloroform-d) δ 158.68; 139.77; 139.46; 134.72; 130.09; 127.53; 126.56; 124.60; 115.78; 104.80; 62.18; 52.86; 52.16.

ESI MS m/z; calculated: 341.0056 C₁₄H₁₄BrClN₂O [M+H]⁺; found: 341.0055 [M+H]⁺.

### EXAMPLE 33

### Preparation of 2-[(4-methylbenzyl)(6-bromo-pyridin-2-yl)amino]ethan-1-ol

2-((6-bromo-pyridin-2-yl)amino)ethan-1-ol (0.0046 mol; 1 g) in 5 mL DMF, 1-(bromomethyl)-4-methylbenzene (0, 0069 mol; 1.26 g), and diisopropylethylamine (0.007 mol; 1.13 mL) were placed in a round bottom flask. The mixture was stirred using a magnetic stirrer and heated to 110° C. in an oil bath under reflux for 24 hours, controlling the reaction by means of a thin layer chromatography. After completion of the reaction, the solvents were evaporated and the product was extracted using DCM/saturated aqueous solution of NaHCO₃ (1/1 v/v). The product was then purified by silica gel column chromatography using hexane/ethyl acetate mixtures as eluents (gradient method, starting with one-component eluent (100% hexane) and ending with the mixture 7/3 v/v as the final eluent), and the solvents were evaporated on a rotary evaporator. To completely remove traces of the organic solvents, the purified product was freeze-dried from benzene. Thus, 990 mg of 2-[(4-methylbenzyl)(6-bromo-pyridin-2-yl)amino]ethan-1-ol were obtained as oil with a yield of 68%. The product was characterized by NMR spectroscopy:

¹H NMR (400 MHz, Chloroform-d) δ 7.22 (dd, J=8.4, 7.5 Hz, 1H, H4); 7.15 (d, J=8.0 Hz, 2H, H10, H14); 7.10 (d, J=8.0 Hz, 2H, H11, H13); 6.75 (d, J=7.4 Hz, 1H, H5); 6.40 (d, J=8.4 Hz, 1H, H3); 4.64 (s, 2H, H9); 3.88 (td, J=4.8; 1.1 Hz, 2H, H8); 3.84-3.77 (m, 2H, H7); 3.15 (s, 1H, —OH); 2.34 (s, 3H, p-CH3),

¹³C NMR (101 MHz, Chloroform-d) δ 158.50 (C2); 139.32 (C4); 138.56 (C6); 136.64 (C12); 133.24 (C15); 129.07 (C10, C14); 125.92 (C11, C13); 115.08 (C5); 104.80 (C3); 62.15 (C8); 52.88 (C9); 52.10 (C7); 20.57 (C16),

ESI MS m/z; calculated: 321.0602 C₁H₁₇BrN₂O [M+H]⁺; found: 321.0600 [M+H]⁺.

### EXAMPLE 34

### Preparation of 2-[(6-bromo-pyridin-2-yl)benzylamino]ethane-1-ol

2-((6-bromo-pyridin-2-yl)amino)ethan-1-ol (0.0046 mol; 1 g) in 5 mL DMF, benzyl bromide (0.0069 mol; 0.83 mL), and diisopropylethylamine (0.007 mol; 1.13 mL) were placed in a round bottom flask. The mixture was stirred using a magnetic stirrer and heated to 110° C. in an oil bath under reflux for 72 hours, controlling the reaction by means of a thin layer chromatography. After completion of the reaction, the solvents were evaporated and the product was extracted using DCM/saturated aqueous solution of NaHCO₃ (1/1 v/v). The product was then purified by silica gel column chromatography using hexane/ethyl acetate mixtures as eluents (gradient method, starting with one-component eluent (100% hexane) and ending with the mixture 7/3 v/v as the final eluent), and the solvents were evaporated on a rotary evaporator. To completely remove traces of the organic solvents, the purified product was freeze-dried from benzene. Thus, 790 mg of 2-[(6-bromo-pyridin-2-yl)benzylamino]ethan-1-ol were obtained as oil with a yield of 55%. The product was characterized by NMR spectroscopy:

¹H NMR (400 MHz, Chloroform-d) δ 7.38-7.20 (m, 6H, 2×H11, 12, 2×10.4); 6.76 (d, J=7.4 Hz, 1H, H5); 6.39 (d, J=8.4 Hz, 1H, H3); 4.69 (s, 2H, H9); 3.93-3.85 (m, 2H, H8); 3.84-3.77 (m, 2H); 3.17 (s, 1H, H7),

¹³C NMR (101 MHz, Chloroform-d) δ 158.53 (C2); 139.29 (C4); 138.74 (C6); 136.49 (C13); 128.38 (C11); 126.91 (C12); 125.95 (C10); 115.12 (C5); 104.66 (C3); 62.11 (C8); 53.05 (C9); 52.05 (C7),

ESI MS m/z; calculated: 307.0446 C₁₄H₁₅BrN₂O [M+H]⁺; found: 307.0443 [M+H]⁺.

### EXAMPLE 35

### Preparation of 3-[(6-bromo-pyridin-2-yl)benzylamino]propan-1-ol

3-((6-bromo-pyridin-2-yl)amino)propan-1-ol (0.0046 mol; 1 g) in 5 mL DMF, benzyl bromide (0.0069 mol; 0.83 mL), and diisopropylethylamine (0.007 mol; 1.13 mL) were placed in a round-bottomed flask. The mixture was stirred using a magnetic stirrer and heated to 110° C. in an oil bath under reflux for 72 hours, controlling the reaction by means of a thin layer chromatography. After completion of the reaction, the solvents were evaporated and the product was extracted using DCM/saturated aqueous solution of NaHCO₃ (1/1 v/v). The product was then purified by silica gel column chromatography using hexane/ethyl acetate mixtures as eluents (gradient method, starting with one-component eluent (100% hexane) and ending with the mixture 7/3 v/v as the final eluent), and the solvents were evaporated on a rotary evaporator. To completely remove traces of the organic solvents, the purified product was freeze-dried from benzene. Thus, 710 mg of 3-[(6-bromo-pyridin-2-yl)benzylamino]propan-1-ol were obtained as oil with a yield of 51%.

### EXAMPLE 36

### Preparation of 2-[(6-bromo-pyridin-2-yl)naphthylamino]ethan-1-ol

2-((6-bromo-pyridin-2-yl)amino)ethan-1-ol (0.0069 mol; 1.5 g) in 5 mL DMF, 1-(bromomethyl)-naphthalene (0.0076 mol; 1.67 g), and diisopropylethylamine (0.007 mol; 1.13 mL) were placed in a round-bottomed flask. The mixture was stirred using a magnetic stirrer and heated to 110° C. in an oil bath under reflux for 24 hours, controlling the reaction by means of a thin layer chromatography. After completion of the reaction, the solvents were evaporated and the product was extracted using DCM/saturated aqueous solution of NaHCO₃ (1/1 v/v). The product was then purified by silica gel column chromatography using hexane/ethyl acetate mixtures as eluents (gradient method, starting with one-component eluent (100% hexane) and ending with the mixture 7/3 v/v as the final eluent), and the solvents were evaporated on a rotary evaporator. To completely remove traces of the organic solvents, the purified product was freeze-dried from benzene. Thus, 1.4 g of 2-[(6-bromo-pyridin-2-yl)naphthylamino]ethan-1-ol was obtained as oil with a yield of 58%. The product was characterized by NMR spectroscopy:

¹H NMR (500 MHz, DMSO-d₆) δ 7.82 (d, J=8.6 Hz, 2H); 7.80-7.76 (m, 1H); 7.61 (s, 1H); 7.52-7.41 (m, 2H); 7.33 (dd, J=8.5; 1.8 Hz, 1H); 7.18 (dd, J=8.4; 7.4 Hz, 1H); 6.74 (d, J=7.4 Hz, 1H); 6.40 (d, J=8.4 Hz, 1H); 4.82 (s, 2H); 3.89 (t, J=5.2 Hz, 2H); 3.84 (dd, J==5.3, 4.1 Hz, 2H),

¹³C NMR (126 MHz, DMSO-d₆) δ 158.98; 139.68; 139.34; 134.46; 133.34; 132.68; 128.69; 127.63; 126.30; 125.83; 124.85; 124.63; 115.50; 105.03; 62.43; 53.55; 52.28, ESI MS m/z; calculated: 357.0602 C₁₈H₁₇BrN₂O [M+H]⁺; found: 379.0422.

### EXAMPLE 37

### Preparation of (E)-2-(benzyl{6-[2-(pyridin-2-yl)vinyl]pyridin-2-yl}amino)ethan-1-ol

Pd(OAc)₂ (0.065 mmol, 15 mg) and PPh₃ (0.19 mmol, 51 mg) dissolved in 5 mL of anhydrous DMF were placed in a round-bottom flask flushed with argon. After 10 min, TBAB (0.05 mmol; 16 mg), 2-[(6-bromo-pyridin-2-yl)benzylamino]ethan-1-ol (1.6 mmol; 500 mg), vinylpyridine (1.9 mmol; 0.21 mL), and Cs₂CO₃ (2.4 mmol, 760 mg) were added. The reaction was carried out under an argon atmosphere in a microwave reactor (120° C., 300 W) for 3 hours. After completion of the reaction, DMF was evaporated and the row product was preliminarily purified by extraction from DCM/saturated aqueous solution of NaHCO₃ (1/1; v/v). The product was then purified by silica gel column chromatography using hexane/ethyl acetate mixtures as eluents (gradient method, starting with one-component eluent (100% hexane) and ending with the mixture 1/1 v/v as the final eluent), and the solvents were evaporated on a rotary evaporator. To completely remove traces of the organic solvents, the purified product was freeze-dried from benzene. Thus, 324 mg of (E)-2-(benzyl{6-[2-(pyridin-2-yl)vinyl]pyridin-2-yl}amino)ethan-1-ol was obtained as oil with a yield of 60%. The product was characterized by NMR spectroscopy:

¹H NMR (500 MHz, Chloroform-d) δ 8.55-8.49 (m, 1H, H6); 7.59 (td, J=7.7; 1.8 Hz, 1H, H4); 7.53 (d, J=15.8 Hz, 1H, H7); 7.44 (d, J=15.9 Hz, 1H, H8); 7.40 (dt, J=7.9; 1.0 Hz, 1H); 7.30 (dd, J=8.5; 7.3 Hz, 1H, H11); 7.25-7.22 (m, 1H); 7.19-7.13 (m, 4H); 7.08 (ddd, J=7.5; 4.8; 1.1 Hz, 1H); 6.67 (d, J=7.2 Hz, 1H); 6.33 (d, J=8.5 Hz, 1H); 4.62 (s, 2H); 3.85 (t, J=2.7 Hz, 4H),

¹³C NMR (126 MHz, Chloroform-d) δ 158.86 (C13); 155.05 (C2); 152.15 (C9); 149.32 (C6); 138.50 (C11); 137.52 (C18); 136.79 (C4); 131.94 (C8); 130.69 (C7); 128.81 (C19); 127.22 (C21); 126.34 (C20); 123.09 (C3); 122.50 (C5); 113.50 (C10); 107.06 (C12); 63.33 (C16); 53.52 (C17); 52.40 (C15),

ESI MS m/z; calculated: 332.1763 C₂₁H₂₁N₃O [M+H]⁺; found: 332.1758 [M+H]⁺.

### EXAMPLE 38

### Preparation of (E)-3-(benzyl{6-[2-(pyridin-2-yl)vinyl]pyridin-2-yl}amino)propan-1-ol

Pd (OAc)₂ (0.065 mmol, 15 mg) and PPh₃ (0.19 mmol, 51 mg) dissolved in 5 mL of anhydrous DMF were placed in a round-bottom flask flushed with argon. After 10 min, TBAB (0.05 mmol; 16 mg), 3-[(6-bromo-pyridin-2-yl)benzylamino]propan-1-ol (1.6 mmol; 485 mg), vinyl pyridine (1.9 mmol; 0, 21 mL), and Cs₂CO₃ (2.4 mmol, 760 mg) were added. The reaction was carried out under an argon atmosphere in a microwave reactor (120° C., 300 W) for 3 hours. After completion of the reaction, DMF was evaporated and the row product was preliminarily purified by extraction from DCM/saturated aqueous solution of NaHCO₃ (1/1; v/v). The product was then purified by silica gel column chromatography using hexane/ethyl acetate mixtures as eluents (gradient method, starting with one-component eluent (100% hexane) and ending with the mixture 1/1 v/v as the final eluent), and the solvents were evaporated on a rotary evaporator. To completely remove traces of the organic solvents, the purified product was freeze-dried from benzene. Thus, 324 mg of (E)-3-(benzyl{6-[2-(pyridin-2-yl)vinyl]pyridin-2-yl}amino)propan-1-ol was obtained as oil with a yield of 60%.

### EXAMPLE 39

### Preparation of (E)-2-(naphthyl-{6-[2-(pyridin-2-yl)vinyl]pyridin-2-yl}amino)ethan-1-ol

Pd (OAc)₂ (0.21 mmol, 47 mg) and PPh₃ (0.63 mmol, 165 mg) in 10 mL of anhydrous DMF were placed in a round-bottom flask flushed with argon. After 10 min, TBAB (0.1 mmol; 32 mg), 2-[(6-bromo-pyridin-2-yl) naphthylamino] ethan-1-ol (4.2 mmol; 900 mg), vinylpyridine (5.0 mmol; 0.54 mL), and Cs₂CO₃ (6.3 mmol, 2 g) were added. The reaction was carried out under an argon atmosphere in a microwave reactor (120° C., 300 W) for 3 hours. After completion of the reaction, DMF was evaporated and the row product was preliminarily purified by extraction from DCM/saturated aqueous solution of NaHCO₃ (1/1; v/v). The product was then purified by silica gel column chromatography using hexane/ethyl acetate mixtures as eluents (gradient method, starting with one-component eluent (100% hexane) and ending with the mixture 1/1 v/v as the final eluent), and the solvents were evaporated on a rotary evaporator. To completely remove traces of the organic solvents, the purified product was freeze-dried from benzene. Thus, 249 mg of (E)-2-(naphthyl-{6-[2-(pyridin-2-yl)vinyl]pyridin-2-yl}amino)ethan-1-ol was obtained as oil with a yield of 28%. The product was characterized by means of mass spectrometry:

ESI MS m/z; calculated: 382.1919 C₂₅H₂₃N₃O [M+H]⁺; found: 382.1915 [M+H]⁺.

### EXAMPLE 40

### Preparation of (E)-2-(2,4-difluoro-6-{[2-(pyridin-2-yl)vinyl]pyridin-2-yl}amino)ethan-1-ol

Pd (OAc)₂ (0.046 mmol, 10 mg) and PPh₃ (0.14 mmol, 36 mg) in 10 mL of anhydrous DMF were placed in a round-bottom flask flushed with argon. After 10 min, TBAB (0.1 mmol; 32 mg), 2-[(2,4-difluorobenzyl)(6-bromo-pyridin-2-yl)amino]-ethan-1-ol (2.33 mmol; 800 mg), vinylpyridine (2.8 mmol; 0.3 mL), and Cs₂CO₃ (2.99 mmol; 971 mg) were added. The reaction 1255 was carried out under an argon atmosphere in a microwave reactor (120° C., 300 W) for 4 hours. After completion of the reaction, DMF was evaporated and the row product was preliminarily purified by extraction from DCM/saturated aqueous solution of NaHCO₃ (1/1; v/v). The product was then purified by silica gel column chromatography using hexane/ethyl acetate mixtures as eluents (gradient method, starting with one-component eluent (100% hexane) and ending with the mixture 1/1 v/v as the final eluent), and the solvents were evaporated on a rotary evaporator. To completely remove traces of the organic solvents, the purified product was freeze-dried from benzene. Thus, 412 mg of (E)-2-(2,4-difluorobenzyl){6-[2-(pyridin-2-yl) vinyl]pyridin-2-yl} amino) ethan-1-ol was obtained as oil in a yield of 48%. The product was characterized by NMR spectroscopy:

¹⁹F NMR (376 MHz, Chloroform-d) δ −111.77 (p, J=7.8 Hz), −114.25 (q, J=8.6 Hz), ESI MS m/z; calculated: 368.1574 C₂₁H₁₉F₂N₃O [M+H]⁺; found: 368.1570 [M+H]⁺.

### EXAMPLE 41

### Preparation of (E)-2-(4-methyl-6-{[2-(pyridin-2-yl)vinyl]pyridin-2-yl}amino)ethan-1-ol

Pd (OAc)₂ (0.025 mmol; 5.6 mg) and PPh₃ (0.075 mmol; 20 mg) dissolved in 5 mL of anhydrous DMF were placed in a round-bottom flask flushed with argon. After 10 min, TBAB (0.05 mmol; 16 mg), 2-[(4-methylbenzyl)(6-bromo-pyridin-2-yl) amino]ethan-1-ol (0.6 mmol; 200 mg), vinylpyridine (0.74 mmol; 81 μL), and Cs₂CO₃ (0.9 mmol; 305 mg) were added. The reaction was carried out under an argon atmosphere in a microwave reactor (120° C., 300 W) for 4 hours. After completion of the reaction, DMF was evaporated and the row product was preliminarily purified by extraction from DCM/saturated aqueous solution of NaHCO₃ (1/1; v/v). The product was then purified by silica gel column chromatography using hexane/ethyl acetate mixtures as eluents (gradient method, starting with one-component eluent (100% hexane) and ending with the mixture 1/1 v/v as the final eluent), and the solvents were evaporated on a rotary evaporator. To completely remove traces of the organic solvents, the purified product was freeze-dried from benzene. Thus, 90 mg of (E)-2-(4-methylbenzyl{6-[2-(pyridin-2-yl)vinyl]pyridin-2-yl}amino)ethan-1-ol were obtained as oil in a yield of 42%.

### EXAMPLE 42

### Preparation of (E)-2-(2-chloro-6-{[2-(pyridin-2-yl)vinyl]pyridin-2-yl}amino)ethan-1-ol

Pd (OAc)₂ (0.023 mmol; 5.3 mg) and PPh₃ (0.07 mmol; 18 mg) dissolved in 5 mL of anhydrous DMF were placed in a round-bottom flask flushed with argon. After 10 min, TBAB (0.05 mmol; 16 mg), 2-[(2-chlorobenzyl)(6-bromo-pyridin-2-yl)amino]ethan-1-ol (0.58 mmol; 200 mg), vinylpyridine (0.7 mmol; 76 μL), and Cs₂CO₃ (0.88 mmol; 287 mg) were added. The reaction was carried out under an argon atmosphere in a microwave reactor (120° C., 300 W) for 4 hours. After completion of the reaction, DMF was evaporated and the row product was preliminarily purified by extraction from DCM/saturated aqueous solution of NaHCO₃ (1/1; v/v). The product was then purified by silica gel column chromatography using hexane/ethyl acetate mixtures as eluents (gradient method, starting with one-component eluent (100% hexane) and ending with the mixture 1/1 v/v as the final eluent), and the solvents were evaporated on a rotary evaporator. To completely remove traces of the organic solvents, the purified product was freeze-dried from benzene. Thus, 88 mg of (E)-2-(2-chlorobenzyl{6-[2-(pyridin-2-yl)vinyl]pyridin-2-yl}amino) ethan-1-ol was obtained as yellow oil with a yield of 41%. The product was characterized by NMR spectroscopy:

¹H NMR (500 MHz, Chloroform-d) δ 8.60 (dd, J=5.3, 1.5 Hz, 1H, H6); 7.67 (td, J=7.7; 1.9 Hz, 1H, H4); 7.61 (d, J=15.8 Hz, 1H, H7); 7.53 (d, J=15.8 Hz, 1H, H8); 7.48 (d, J=7.8 Hz, 1H, H3); 7.40 (dd, J=8.5, 7.2 Hz, 1H, H11); 7.27-7.21 (m, 3H, H21, 22, 23); 7.17 (ddd, J=7.5; 4.8; 1.1 Hz, 1H, H5); 7.13 (dt, J=7.1; 1.7 Hz, 1H, H2O); 6.77 (d, J=7.2 Hz, 1H, H10); 6.38 (d, J=8.5 Hz, 1H, H12); 4.69 (s, 2H, H17); 3.94 (dd, J=5.2; 3.8 Hz, 2H, H16); 3.90 (dd, J=7.5, 3.0 Hz, 2H, H15),

¹³C NMR (126 MHz, Chloroform-d) δ 158.54 (C13); 155.00 (C2); 152.31 (C9); 149.31 (C6); 140.07 (C18); 138.53 (C11); 136.76 (C4); 134.72 (C19); 131.96 (C8); 130.73 (C7); 130.08 (C23); 127.40 (C21); 126.48 (C22); 124.51 (C20); 123.04 (C3); 122.49 (C5); 113.68 (C10); 106.75 (C12); 62.91 (C16); 52.96 (C17); 52.18 (C15),

ESI MS in/z; calculated: 366.1373 C₂₁H₂₀ClN₃O [M+H]⁺; found: 366.1372 [M+H]⁺.

### EXAMPLE 43

### Preparation of (E)-2-{(2,4-dimethylbenzyl)[6-((2-pyridin-2-yl)vinyl] pyridin-2-yl)amino}ethan-1-ol

Pd (OAc)₂ (0.021 mmol; 4.8 mg) and PPh₃ (0.064 mmol; 17 mg) dissolved in 5 mL of anhydrous DMF were placed in a round-bottom flask flushed with argon. After 10 min, TBAB (0.05 mmol; 16 mg), 2-{2,4-dimethylbenzyl-(6-bromopyridin-2-yl)}-aminoethan-1-ol (0.54 mmol; 180 mg), vinylpyridine (0, 64 mmol; 70 μL), and Cs₂CO₃ (0.8 mmol; 262 mg) were added. The reaction was carried out under an argon atmosphere in a microwave reactor (120° C., 300 W) for 4 hours. After completion of the reaction, DMF was evaporated and the row product was preliminarily purified by extraction from DCM/saturated aqueous solution of NaHCO₃ (1/1; v/v). The product was then purified by silica gel column chromatography using hexane/ethyl acetate mixtures as eluents (gradient method, starting with one-component eluent (100% hexane) and ending with the mixture 1/1 v/v as the final eluent), and the solvents were evaporated on a rotary evaporator. To completely remove traces of the organic solvents, the purified product was freeze-dried from benzene. Thus, 83 mg of (E)-2-{(2,4-dimethylbenzyl)[6-((2-pyridin-2-yl)vinyl]pyridin-2-yl)amino}ethan-1-ol was obtained as oil in a yield of 43%. The product was characterized by mass spectrometry:

ESI MS m/z; calculated: 360.206 C₂₃H₂₅N₃O [M+H]⁺; found: 360.2071 [M+H]⁺.

### EXAMPLE 44

### Preparation of (E)-2-{2-fluorobenzyl-[6-((2-pyridin-2-yl)vinyl)pyridin-2-yl)-aminoethan-1-ol

Pd(OAc)₂ (0.025 mmol; 5.6 mg) and PPh₃ (0.075 mmol; 20 mg) dissolved in 5 mL of anhydrous DMF were placed in a round-bottom flask flushed with argon. After 10 min, TBAB (0.05 mmol; 16 mg), 2-[(2-fluorobenzyl)(6-bromo-pyridin-2-yl)amino]ethan-1-ol (0.61 mmol; 200 mg), vinylpyridine (0.73 mmol; 79 μL), and Cs₂CO₃ (0.9 mmol; 300 mg) were added. The reaction was carried out under an argon atmosphere in a microwave reactor (120° C., 300 W) for 4 hours. After completion of the reaction, DMF was evaporated and the row product was preliminarily purified by extraction from DCM/saturated aqueous solution of NaHCO₃ (1/1; v/v). The product was then purified by silica gel column chromatography using hexane/ethyl acetate mixtures as eluents (gradient method, starting with one-component eluent (100% hexane) and ending with the mixture 1/1 v/v as the final eluent), and the solvents were evaporated on a rotary evaporator. To completely remove traces of the organic solvents, the purified product was freeze-dried from benzene. Thus, 75 mg of (E)-2-{2-fluorobenzyl-[6-((2-pyridin-2-yl)vinyl)pyridin-2-yl)aminoethan-1-ol was obtained as yellow oil in a 35% yield. The product was characterized by NMR spectroscopy:

¹H NMR (500 MHz, Chloroform-d) δ 8.61 (dd, J=5.0, 1.7 Hz, 1H, H6); 7.66 (td, J=7.7; 1.8 Hz, 1H, H4); 7.60 (d, J=15.7 Hz, 1H, H7); 7.53 (d, J=15.7 Hz, 1H, H8); 7.46 (d, J=7.8 Hz, 1H, H3); 7.40 (dd, J=8.5; 7.3 Hz, 1H, H11); 7.27-7.22 (m, 1H, H21); 7.23-7.14 (m, 2H, H5, H23); 7.12-7.04 (m, 2H, H2O, 22); 6.77 (d, J=7.2 Hz, 1H, H10); 6.40 (d, J=8.5 Hz, 1H, H12); 4.76 (s, 2H, H17); 4.03-3.81 (m, 4H, H15, 16),

¹³C NMR (126 MHz, Chloroform-d) δ 160.81 (C19) (d, JF-C=245.5 Hz); 158.67 (C13); 155.12 (C2); 152.39 (C9); 149.51 (C6); 138.50 (C11); 136.60 (C4); 131.84 (C8); 130.87 (C7); 128.75 (C23); 127.87 (C21); 124.50 (C18); 124.31 (C22); 123.10 (C3); 122.46 (C5); 115.39 (C20); 113.57 (C10); 106.68 (C12); 63.28 (C16); 52.36 (C15); 47.52 (C17),

ESI MS m/z; calculated: 350.1169 C₂₁H₂₀FN₃O [M+H]⁺; found: 360.1164 [M+H]⁺.

### EXAMPLE 45

Labeling of 3-O-Acetylthymidine with a Fluorescent Dye Derivative

### Step 1

Conversion of an Aminoalcohol Derivative of 2-(pyridine-2-yl) vinyl] Pyridine into a Carbamate Derivative

(E)-2-(methyl{6-[2-(pyridin-2-yl)vinyl]pyridin-2-yl}amino)ethan-1-ol (1 eq., 0.1 mmol) was dissolved in anhydrous acetonitrile (2 ml) and placed in a flask containing a magnetic stir bar. To this solution, 1′1′-carbonyldiimidazole (2 eq., 0.24 mmol) was added and the reaction was carried out for 30 minutes at room temperature and its course was monitored by TLC (ethyl acetate/n-hexane 9/1). After 1 hour, the active carbamate derivative of the amino alcohol was obtained, which was used in the next step.

### Step 2

Labeling of 3′-O-Acetylthymidine with a Carbamate Derivative of a Fluorescent Dye

To the solution of carbamate derivative of (E)-2-(methyl(6-(2-(pyridin-2-yl)vinyl)pyridin-2-yl)amino)ethanol obtained in the previous step, 3′-O-acetylthymidine (1 eq; 0.1 mmol) and 1,1,3,3-tetramethylguanidine (1.6 eq., 0.16 mmol) were added. The reaction mixture was stirred for 2 hours at room temperature. The course of the reaction was monitored by TLC (dichloromethane/methanol 95/5 v/v). The solvent was evaporated under reduced pressure and the thus obtained residue was purified by silica gel column chromatography (DCM/MeOH; 0-5% MeOH) to obtain a 3′-O-acetylthymidine derivative labeled with (E)-2-(methyl (6-(2-(pyridin-2-yl) vinyl) pyridin-2-yl) amino) ethanol which was characterized by spectroscopic methods.

¹H NMR (400 MHz; Chloroform-d) δ 8.66-8.55 (m; 1H); 7.70-7.65 (m; 1H); 7.63 (d; J=5.3 Hz; 1H); 7.48 (d; J=8.6 Hz; 1H); 7.44 (d; J=8.0 Hz; 2H); 7.35 (d; J=2.2 Hz; 2H); 7.14 (dd; J=7.5; 4.9 Hz; 1H); 6.71 (d; J=7.3 Hz; 1H); 6.45 (d; J=8.5 Hz; 1H); 6.35 (dd; J=8.8; 5.7 Hz; 1H); 5.16 (dd; J=5.2; 3.2 Hz; 1H); 4.52 (dt; J=11.5; 5.9 Hz; 1H); 4.42 (q; J=4.6; 3.7 Hz; 1H); 4.39 (d; J=2.9 Hz; 1H); 4.33 (dd; J=11.8; 2.8 Hz; 1H); 4.17 (q; J=2.7 Hz; 1H); 4.02 (tq; J=14.7; 8.8; 7.4 Hz; 2H); 3.10 (s; 3H); 2.40-2.29 (m; 1H); 2.22-2.10 (m; 2H); 2.09 (s; 3H); 1.88 (s; 3H). ¹³C NMR (101 MHz; Chloroform-d) δ 169.89; 163.00; 157.25; 155.08; 154.11; 152.11; 149.93; 149.18; 137.44; 136.01; 134.34; 132.11; 130.24; 127.82; 122.10; 121.78; 112.34; 111.15; 104.99; 84.04; 81.62; 74.07; 66.76; 65.86; 47.87; 36.73; 36.64; 20.39; 12.13.

Fluorescence properties (Absorption and emission were measured in methanol; the analyte concentration was in the order of 10⁻⁵-10⁻⁶ M; the measurement error margin for ε is +/−20%)

| | | ε |
|---|---|---|
| λ_{abs} | λₑₘ | [M⁻¹ cm⁻¹] |
| 371 | 485 | 16800 |

1. A method for fluorescence labeling of nucleosides, nucleotides and oligonucleotides with fluorescent dyes wherein a moiety of general formula 1 or 2 or 3 or 4 with the double bond configuration E is attached to a nucleoside, nucleotide or oligonucleotide 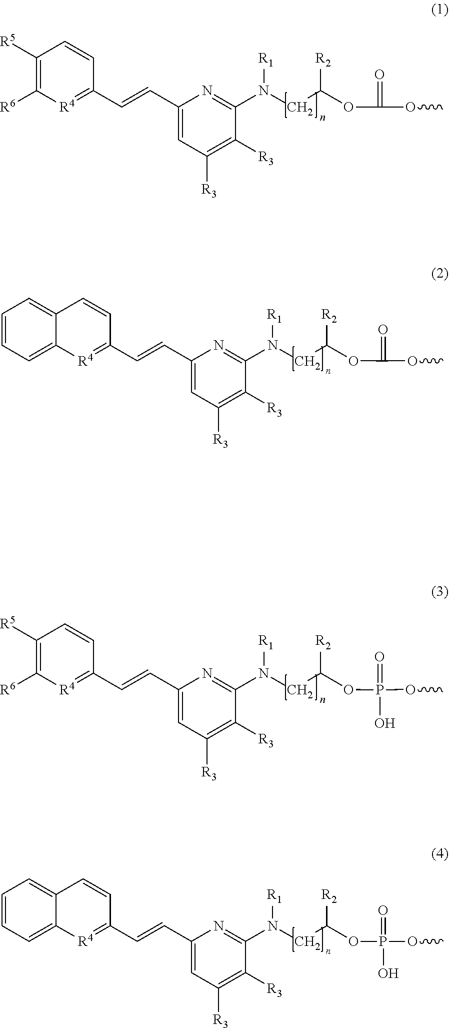
wherein:
n is 1 or 2 [symbol]
—marks the bonding site with the nucleoside or nucleotide or oligonucleotide
R₁ means:
hydrogen, methyl, amino group, carbonyl, benzyl, naphthyl, naphthylmethyl, phenyl, benzyl, quinolinemethyl,
benzyl substituted with one or more the same or different substituents: chlorine, fluorine, methyl,
saturated or unsaturated alkyl containing from 2 to 8 carbon atoms and one double bond, saturated alkyl containing from 2 to 8 carbon atoms substituted with a phenyl, amino group, hydroxyl group or simultaneously phenyl and hydroxyl group or phenyl and amino group,
R₂ means: hydrogen, methyl, benzyl, phenyl, naphthyl, saturated or unsaturated alkyl containing from 2 to 8 carbon atoms and one double bond,
R₃ are the same or different and represent hydrogen, methyl, saturated or unsaturated alkyl containing from 2 to 8 carbon atoms and one double bond, amino group, nitro group, azido group, a group of general formula 5 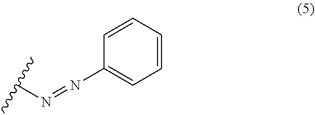
R₄ means carbon or nitrogen
R₅ and R₆ are the same or different and represent hydrogen, methyl, benzyl, phenyl, naphthyl, halogen (F, Cl, Br, I), hydroxyl, amino group, nitro group, carboxyl, saturated or unsaturated alkyl containing 2 to 8 carbon atoms and one double bond, saturated or phenyl substituted unsaturated alkyl containing 2 to 8 carbon atoms and one double bond,
in a chemical reaction of a free primary hydroxyl group of the nucleoside, nucleotide or oligonucleotide with a compound of general formula 6 or 7 or 8 or 9 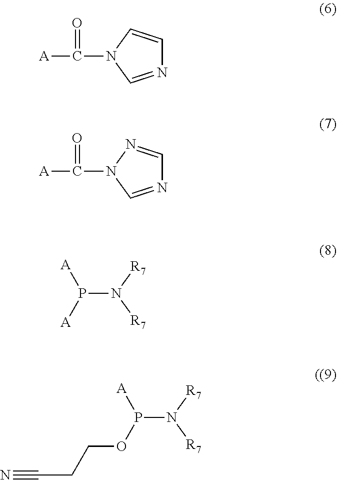
wherein A represents a group of general formula 10 or 11 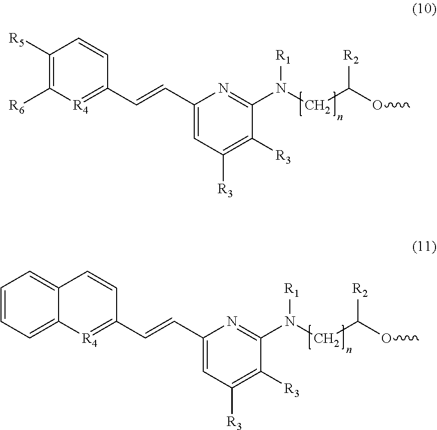
wherein n and substituents R₁, R₂, R₃, R₄, R₅, and R₆ have the same meanings as given above, with the compounds of the formulas 6 and 7 being obtained directly in the reaction medium as reaction products between an alcohol of general formula 12 or 13 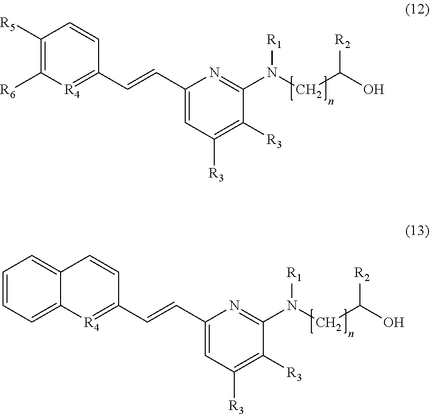
wherein n and substituents R₁, R₂, R₃, EC, R₅, and R₆ have the same meanings as above, with a carbonyldimidazole of general formula 14 or a carbonyldi(1,2, 4-triazole) of general formula 15 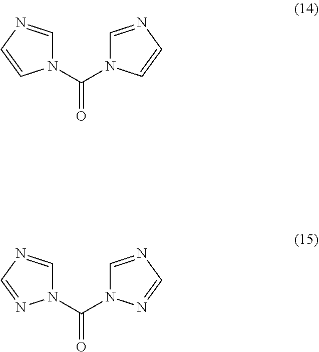
forming an intermediate of general formula 6 or 7, with the compound of formula 8 being added into the reaction medium or being obtained directly in the reaction medium by reacting an alcohol of general formula 12 or 13 wherein n and substituents R₁, R₂, R₃, R₄, R₅, and R₆ have the same meaning as above, with N,N,N′,N′-bis(diisopropylamino)chlorophosphine of formula 16, 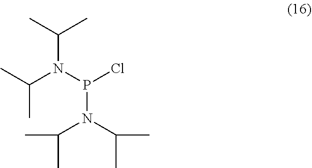
whereat the reaction proceeds in two steps, with first step in which an alcohol of general formula 12 or 13 reacts with N,N,N′,N′-bis(diisopropylamino)chlorophosphine and the second step in which 1-H-tetrazole or 2-ethylthiotetrazole or 4,5-dicyanoimidazole or 5-benzyl-mercaptotetrazole is added in order to enable further metathesis resulting in a product that reveals rests A introduced in reaction with the alcohol of formula 12 or 13, and whereat the compound of formula 9 is injected into the reaction medium or is obtained directly in the reaction medium by reacting an alcohol of formula 12 or 13, wherein n and substituents R₁, R₂, R₃, R₄, R₅, and R₆ have the same meaning as above, with 2-cyanoethyl-N,N,N′,N′-bis(diisopropylamino)phosphine of formula 17 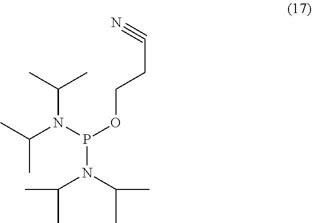 [SMILES: CC(C)N(C(C)C)P(OCCC#N)N(C(C)C)C(C)C]
in the presence of 1H-tetrazole or 2-ethylthiotetrazole or 4,5-dicyanoimidazole or 5-benzyl-mercaptotetrazole, wherein after the reaction between a nucleoside, nucleotide or oligonucleotide and the intermediate of formula 8 or 9 the reaction product is subjected to standard agents used in the oxidation of phosphorus (III) compounds, whereat in the case when CSO is used, methylamine or 1,8-diazabicyclo[5.4.0]undec-7-ene (DBU) or concentrated aqueous ammonia solution is added after the oxidation step is completed.

2. The method according to claim 1 wherein all reactions are carried out at a temperature not higher than 30° C.

3. The method according to claim 1 wherein the reaction between alcohol of formula 12 or 13 and carbonyldiimidazole (14) or karbonyldi(1,2, 4-triazole) (15) is carried out in an anhydrous polar aprotic organic solvent selected from the group consisting of acetonitrile, dimethyl sulfoxide, dimethylformamide, aliphatic ethers, in an inert atmosphere, wherein carbonyldimidazole or carbonylditriazole is used in a proportion of 1 to 1.5 equivalent, relative to the alcohol of formula 12 or 13.

4. The method according to claim 1 wherein after the reaction between alcohol of formula 12 or 13 and carbonyldiimidazole (14) or carbonyldi(1,2, 4-triazole) (15) into the reaction mixture a nucleoside, nucleotide or oligonucleotide is added in a ratio of 1-2 eq. relative to the alcohol of general formula 12 or 13.

5. The method according to claim 4 wherein the reaction is carried out with an addition of a non-nucleophilic organic base selected from the group consisting of guanidine derivatives having the same or different substituents selected from methyl, ethyl, isopropyl; in an amount of 0.3-7.8 eq., relative to the alcohol of general formula 12 or 13.

6. The method according to claim 1 wherein the reaction between the alcohol of formula 12 or 13 and N,N,N′,N′-bis(diisopropylamino)chlorophosphine (16) is carried out in anhydrous acetonitrile or methylene chloride in an atmosphere of inert gas, using N,N,N′,N′-bis(diisopropylamino)chlorophosphine (16) in an amount of 0.4 to 0.6 eq., relative to the alcohol 12 or 13, wherein the reaction is carried out in two steps whereat in the first step, N,N,N′,N′-bis(diisopropylamino)chlorophosphine reacts with one molecule of alcohol of formula 12 or 13 and then in the second step 1-H-tetrazole or 2-ethylthiotetrazole or 4,5-dicyanoimidazole or 5-benzyl-mercaptotetrazole is added in an amount of 0.3 to 0.8 eq. relative to the alcohol of formula 12 or 13 as an activator which initiates the reaction with a second alcohol molecule of formula 12 or 13 and the reaction is continued until completion.

7. The method according to claim 1 wherein the reaction between the alcohol 12 or 13 and 2-cyanoethyl-N,N,N′,N′-bis(diisopropylamino)phosphine (17) is carried out in acetonitrile or methylene chloride under an inert gas atmosphere, in the presence of 1H-tetrazole or 2-ethylthiotetrazole or 4,5-dicyanoimidazole or 5-benzyl-mercaptotetrazole used in an amount of 0.5 to 1 eq. and wherein 2-cyanoethyl-N,N,N′,N′-bis(diisopropylamino)phosphine (17) is used in an amount of 0.8 to 1.1 eq. relative to the alcohol.

8. The method according to claim 1 wherein the proportion of the mixture of compound 8 or 9 and 1H-tetrazole or 2-ethylthiotetrazole or 4,5-dicyanoimidazole or 5-benzyl-mercaptotetrazole, relative to the nucleoside, nucleotide or oligonucleotide for reactions carried out in the liquid phase is 2 eq. to 1 eq.

9. The method according to claim 1 wherein the proportion of the mixture of compounds 8 or 9 and 1H-tetrazole or 2-ethylthiotetrazole or 4,5-dicyanoimidazole or 5-benzyl-mercaptotetrazole relative to the nucleoside, nucleotide or oligonucleotide for solid-phase reactions is 20 eq. to 1 eq.

10. A method of thermal detachment of fluorescent labels from labeled nucleosides, nucleotides or oligonucleotides labeled according to a method revealed in claim 1, wherein the nucleoside, nucleotide or oligonucleotide labeled with a thermally removable label of formula 1 or 2 or 3 or 4 is dissolved in a phosphate buffer (pH in the range from 6.86 to 7.2) or a mixture of phosphate buffer and acetonitrile, and heated to a temperature in the range of 30-90° C.

11. A method of monitoring the progress of thermal detachment of fluorescent labels from a labeled nucleoside, nucleotide or oligonucleotide wherein a difference in fluorescence intensity at the wavelength range corresponding to the label emission is determined between the fluorescence of a dissolved heated sample of a labeled nucleoside or nucleotide or oligonucleotide and a respective sample at lower temperature, wherein both samples are measured at the same time intervals and then the data obtained are analyzed numerically.

12. Pyridin-2-yl-vinylopyridine derivatives of general formula 12 or 13 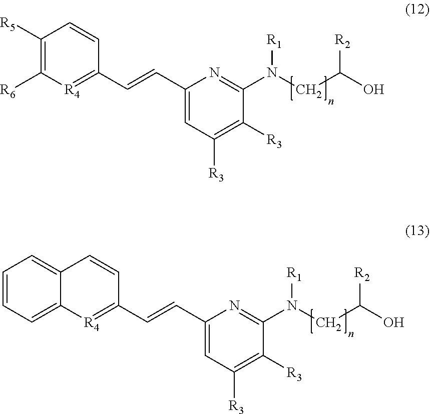
wherein:
n is 1 or 2
R₁ means:
hydrogen, methyl, amino group, carbonyl, benzyl, naphthyl, naphthylmethyl, phenyl, benzyl, quinolinemethyl,
benzyl substituted with one or more the same or different substituents: chlorine, fluorine, methyl,
saturated or unsaturated alkyl containing from 2 to 8 carbon atoms and one double bond, saturated alkyl containing from 2 to 8 carbon atoms substituted with a phenyl, amino group, hydroxyl group or simultaneously phenyl and hydroxyl group or phenyl and amino group,
R₂ means: hydrogen, methyl, benzyl, phenyl, naphthyl, saturated or unsaturated alkyl containing from 2 to 8 carbon atoms and one double bond,
R₃ are the same or different and represent hydrogen, methyl, saturated or unsaturated alkyl containing from 2 to 8 carbon atoms and one double bond, amino group, nitro group, azido group, a group of general formula 5, 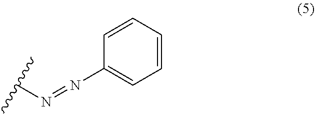
R₄ means carbon or nitrogen,
R₅ and R₆ are the same or different and represent hydrogen, methyl, benzyl, phenyl, naphthyl, halogen (F, Cl, Br, I), hydroxyl, amino group, nitro group, carboxyl, saturated or unsaturated alkyl containing 2 to 8 carbon atoms and one double bond, saturated or phenyl substituted unsaturated alkyl containing 2 to 8 carbon atoms and one double bond.

13. A preparation method of compounds of general formula 12 or 13, wherein n, R₁, R₂, R₃, R₄, R₅, and R₆ have the same meaning as given above, consisting in a reaction between a compound of formula 26 or formula 27 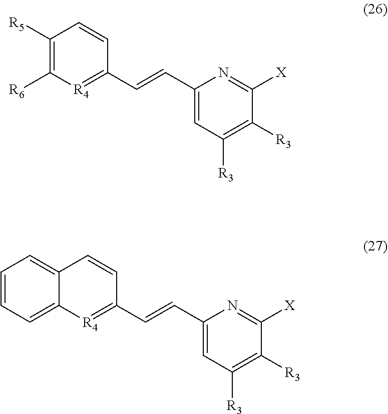
wherein R₃, R₄, R₅, and R₆ have the same above meaning as given above, with an aminoalcohol of general formula 28 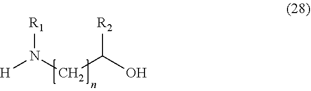
wherein n, R₁, and R₂ have the same meaning as given above,
in the presence of a tertiary organic amine in an anhydrous aprotic polar organic solvent.

14. A preparation method of pyridin-2-yl-vinylpyridine derivatives of general formula 12 or 13 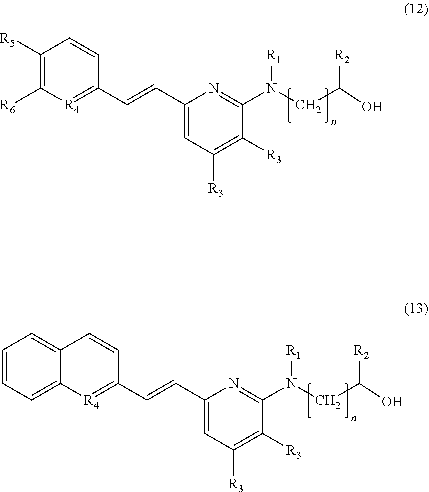
wherein:
n is 1 or 2
R₄ means:
hydrogen, methyl, naphthyl, naphthylmethyl, phenyl, benzyl, quinolinemethyl,
benzyl substituted with one or more the same or different substituents: chlorine, fluorine, methyl,
R₂ means: hydrogen, methyl, benzyl, phenyl, naphthyl, saturated or unsaturated alkyl containing from 2 to 8 carbon atoms and one double bond,
R₃ are the same or different and represent hydrogen, methyl, saturated or unsaturated alkyl containing from 2 to 8 carbon atoms and one double bond, amino group, nitro group, azido group, a group of general formula 5, 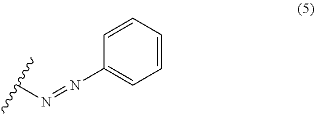
R₄ means carbon or nitrogen,
R₅ and R₆ are the same or different and represent hydrogen, methyl, benzyl, phenyl, naphthyl, halogen (F, Cl, Br, I), hydroxyl, amino group, nitro group, carboxyl, saturated or unsaturated alkyl containing 2 to 8 carbon atoms and one double bond, saturated or phenyl substituted unsaturated alkyl containing 2 to 8 carbon atoms and one double bond, consists in a reaction between a compound of general formula 29 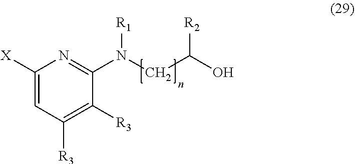
wherein R₁, R₂, and R₃ have the same meaning as given above and X is halogen (Br, Cl, I) with a compound of general formula 30 or 31 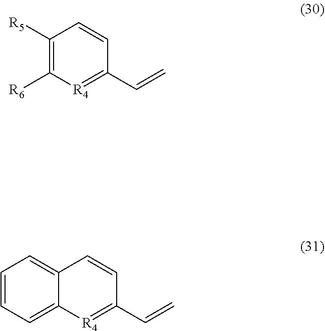
wherein R₅ and R₆ have the same meanings as given above and Rₜ means carbon or nitrogen in the presence of palladium (II) salts, wherein the reaction is carried out in a polar aprotic organic solvent selected from the group consisting of acetonitrile, dimethyl sulfoxide, dimethylformamide, aliphatic ethers.

15. The method according to claim 14 wherein into the polar aprotic organic solvent, a palladium (II) salt, in an amount of 0.01 eq. up to 0.1 eq. and a phosphine, in an amount of 0.03 eq. to 0.3, are added and subsequently a compound 30 or 31 in an amount of 0.6 eq. to 1.5 eq., or 0.6 eq. to 1.5 eq., respectively, a compound of formula 29, 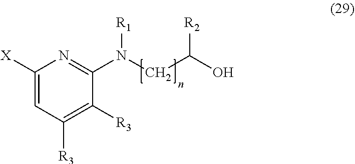
and a base selected from the group consisting of tBuOLi, tBuOK, CS₂CO₃, K₂CO₃, TEA, DBU, KOH, NBu₄OH, NaOAc, K₃PO₄, in an amount of 1.1 eq. up to 1.8 are added.

16. The method according to claim 15 wherein a ionic liquid, tetrabutylammonium acetate (TBAA), 1-butyl-3-methylimidazolium hexafluorophosphate [BMIM][PF6], 1-butyl-3-methylimidazolium bromide [BMIM][Br], tetraheptylammonium bromide (THeptAB) in an amount of 0.03 eq. to 0.1 eq., relative to the palladium salt is applied.
